(19) United States
(12) Patent Application Publication
(10) Pub. No.: US 2026/0224692 A1
(43) Pub. Date: Aug. 6, 2026

(54) T CELL-DIRECTED ANTI-AGING AND ANTI-CANCER VACCINES AGAINST COMMENSAL CYTOMEGALOVIRUS

(71) Applicant: The General Hospital Corporation, Boston, MA (US)

(72) Inventors: Shadmehr Demehri, Charlestown, MA (US); Tatsuya Hasegawa, Kyoto (JP)

(21) Appl. No.: 19/150,659

(22) PCT Filed: Jan. 25, 2024
(86) PCT No.: PCT/US2024/012995

§ 371 Date: Jul. 24, 2025

Related U.S. Application Data
(60) Provisional application No. 63/441,390, filed on Jan. 26, 2023.

(51) Int. Cl. A61K 39/245; A61K 31/192; A61K 39/00

(52) U.S. Cl. CPC A61K 39/245; A61K 31/192; A61K 2039/5254; A61K 2039/57; A61K 2039/585

(57) ABSTRACT

Immune-based approaches to reduce the risk of, delay the onset of, and/or slow the progression of aging and aging-associated diseases by boosting T cell immunity against commensal human cytomegalovirus (HCMV).

### CROSS-REFERENCE TO RELATED APPLICATIONS

This application claims the benefit of U.S. Provisional Application Ser. No. 63/441,390, filed on Jan. 26, 2023. The entire contents of the foregoing are incorporated herein by reference.

### TECHNICAL FIELD

Immune-based approaches to reduce the risk of, delay the onset of, and/or slowing progression of aging and aging-associated diseases by boosting T cell immunity against commensal human cytomegalovirus (HCMV).

### BACKGROUND

Senescent cells, which develop in response to cellular stress, exhibit irreversible arrest in proliferation while resisting death and can accumulate in the body with age. See, for example, He & Sharpless, Cell, 169, 1000-1011, doi:10.1016/j.cell.2017.05.015 (2017) and Di Micco et al. Nat Rev Mol Cell Biol 22, 75-95, doi:10.1038/s41580-020-00314-w (2021). Despite their permanent cell cycle arrest, senescent cells are not inert. They actively communicate with their surroundings and influence the tissue microenvironment through multiple secretary molecules including pro-inflammatory cytokines and tissue-remodeling factors that are collectively termed the senescence-associated secretory phenotype (SASP). See, for example, Mahmoudi & Brunet Nat Cell Biol 21, 32-43, doi:10.1038/s41556-018-0206-0 (2019). Thus, senescent cells can induce a chronic inflammatory state in tissues, which leads to the development of cancer and aging-associated degenerative disorders.

### SUMMARY

Senescent cell accumulation has been implicated in the pathogenesis of aging-associated diseases including cancer. The mechanism that prevents the accumulation of senescent cells in an aging organ is unclear. As shown herein, a commensal virus-immune axis controls accumulation of senescent fibroblasts in human skin. Senescent fibroblasts were increased in old skin compared with young skin. However, they did not increase with advancing age in the elderly. Increased CXCL9 and cytotoxic CD4⁺ T cell (CD4 CTL) recruitment were significantly associated with reduced levels of senescent fibroblasts in old skin. Senescent fibroblasts expressed human leukocyte antigen class II (HLA-II) and human cytomegalovirus glycoprotein B (HCMV-gB), becoming direct CD4 CTL targets. Skin-resident CD4 CTLs eliminated HCMV-gB⁺ senescent fibroblasts in an HLA-II-dependent manner, and HCMV-gB protein activated CD4 CTLs from the human skin. Collectively, these findings demonstrate HCMV reactivation in senescent cells, which CD4 CTLs can directly eliminate through the recognition of the HCMV-gB antigen. Also, the immune factor, CXCL9, that control the recruitment of cytotoxic CD4⁺ T cells, and short chain fatty acids (SCFAs), butyrate and pentanoate, that boost their function for clearance of senescent cells were identified. SCFAs are known to be metabolites from microbiota, so a vaccine approach that includes HCMV-gB plus adjuvants (e.g., SCFAs) will boost the function of cytotoxic CD4⁺ T cells, and can be used to reduce the risk of, delay the onset of, slowing progression of and/or treat aging, cancer, and other age-related diseases.

Thus, provided herein are methods of delaying the onset or slowing progression of aging in a subject. Also provided herein are methods of reducing the level of senescent fibroblasts in a subject. Also disclosed herein are methods of treating, or reducing the risk of developing, an aging-associated disease or condition in a subject. In some cases, the any of methods described herein include administering to the subject an effective amount of a composition. In some cases, the composition includes a plurality of (i) antigenic proteins from commensal human cytomegaloviruses (HCMV), (ii) antigenic peptides derived from proteins from commensal human cytomegaloviruses, or (iii) live or live-attenuated commensal human cytomegaloviruses; and a T cell adjuvant that increases T cell response to the plurality of the antigenic proteins, the antigenic peptides, or the live or live-attenuated commensal human cytomegaloviruses. In some cases, the level of senescent fibroblasts is reduced in the skin of the subject.

In some cases, the subject has an aging-associated disease. In some cases, the aging-associated disease is one or more of a cancer, a cardiovascular disease, a neurodegenerative disease, a renal disease, an autoimmune disease, arthritis, osteoporosis, macular degeneration, chronic obstructive pulmonary disease (COPD), glaucoma, obesity, fibrosis, interstitial lung disease, cirrhosis, hepatic steatosis, and diabetes. In some cases, the cancer is one or more of osteosarcoma, breast cancer, prostate cancer, colorectal cancer, lung cancer, melanoma, kidney cancer, lymphoma, uterine cancer, pancreatic cancer, non-melanoma skin cancer, and bladder cancer. In some cases, the neurodegenerative disease is one or more of dementia, ataxia, Huntington's disease, a motor neuron disease, or a tau-mediated neurodegenerative disease, optionally Alzheimer's disease, Parkinson's disease, or progressive supranuclear palsy. In some cases, the cardiovascular disease is one or more of atherosclerosis, idiopathic pulmonary fibrosis, coronary heart disease, congestive heart failure, coronary artery disease, peripheral arterial disease, valvular heart disease, arrhythmia, ischemic cardiomyopathy, hypertension, and stroke. In some cases, the autoimmune disease is one or more of multiple sclerosis, Crohn's disease, rheumatoid arthritis, an antineutrophilic cytoplasmic antibody (ANCA) associated vasculitide, and systemic lupus erythematosus. In some cases, the fibrosis is one or more of systemic sclerosis, scleroderma, idiopathic pulmonary fibrosis, and interstitial lung disease.

In some cases, at least one of the antigenic proteins, if present, is a HCMV glycoprotein B. In some cases, at least one of the antigenic proteins, if present, comprises a sequence from HCMV glycoprotein B. In some cases, the sequence from HCMV glycoprotein B is or comprises a truncated sequence of HCMV glycoprotein B. In some cases, each antigenic peptide, if present, comprises a sequence of 9-30 amino acids derived from a protein from a human cytomegalovirus. In some cases, each antigenic peptide, if present, comprises a sequence of 9-30 amino acids derived from HCMV glycoprotein B. In some cases, the commensal human cytomegaloviruses, if present, comprise one or more of the AD169, Towne, Toledo, PH, TR, FIX, VR1814, Merlin, and TB40/E HCMV strains.

In some cases, the T cell adjuvant comprises a short chain fatty acid. In some cases, the short chain fatty acid comprises butyrate, pentanoate, or a combination thereof. In some cases, the T cell adjuvant comprises one or more of nanoparticles that enhance T cell response, poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminum salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).

Also disclosed herein are compositions including a plurality of (i) antigenic proteins from commensal human cytomegaloviruses (HCMV), (ii) antigenic peptides derived from proteins from commensal human cytomegaloviruses, or (iii) live or live-attenuated commensal human cytomegaloviruses; and a T cell adjuvant that increases T cell response to the plurality of the antigenic proteins, the antigenic peptides, or the live or live-attenuated commensal human cytomegaloviruses, for use in a method of treating, or reducing the risk of developing, an aging-associated disease or condition in a subject. In some cases, the aging-associated condition is one or more of graying hair, hearing loss, a cataract, frailty, and sarcopenia. In some cases, the aging-associated disease is one or more of a cancer, a cardiovascular disease, a neurodegenerative disease, a renal disease, an autoimmune disease, arthritis, osteoporosis, macular degeneration, chronic obstructive pulmonary disease (COPD), glaucoma, obesity, fibrosis, cirrhosis, hepatic steatosis, and diabetes. In some cases, each antigenic protein, if present, is a HCMV glycoprotein B. In some cases, each antigenic protein, if present, comprises a sequence from HCMV glycoprotein B. In some cases, the sequence from HCMV glycoprotein B is a truncated sequence of HCMV glycoprotein B. In some cases, each antigenic peptide, if present, comprises a sequence of 9-30 amino acids derived from a protein from a human cytomegalovirus. In some cases, each antigenic peptide, if present, comprises a sequence of 9-30 amino acids derived from HCMV glycoprotein B. In some cases, the commensal human cytomegaloviruses, if present, comprise one or more of the AD169, Towne, Toledo, PH, TR, FIX, VR1814, Merlin, and TB40/E HCMV strains. In some cases, the T cell adjuvant comprises a short chain fatty acid. In some cases, the short chain fatty acid comprises butyrate, pentanoate, or a combination thereof. In some cases, the T cell adjuvant comprises one or more of nanoparticles that enhance T cell response, poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminum salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1G. Senescent cell accumulation in the old human skin is not correlated with increasing age. (A) Representative immunofluorescence (IF) staining of p16^{INK4a} in young and old human skin samples. Arrowheads point to p16^{INK4a}-positive cells in the epidermis and arrows point to p16^{INK4a}-positive cells in the dermis. (B and C) Quantification of p16^{INK4a}-positive senescent cells in the epidermis (B) and dermis (C) per high-power field (hpf) (Mann-Whitney U test). (D) Representative IF staining of p16^{INK4a} and vimentin in young and old skin samples. Arrows point to p16^{INK4a+} vimentin⁺ fibroblasts in the dermis. (E) Quantification of p16^{INK4a+} vimentin⁺ fibroblasts per hpf (Mann-Whitney U test). (F) Correlation between the number of dermal senescent cells and age across young and old skin samples (Student's t test for the Pearson correlation coefficient). (G) Correlation between the number of dermal senescent cells and age within the old skin samples (Student's t test for the Pearson correlation coefficient). Nuclei are stained with 40,6-diamidino-2-phenylindole (DAPI). Dotted lines in IF images mark the epidermal basement membrane. Cells are counted blindly and averaged across 10 randomly selected hpf per skin sample. Bar graphs show mean±SD. n=23 in young and n=31 in the old skin group. Scale bars, 100 μm.

FIGS. 2A-21. Senescent fibroblasts constitute most of the senescent cells in the dermis of human skin. (A) Vimentin-positive/negative dermal p16^{INK4a+} cells in each young and old human skin sample. (B) Representative immunofluorescence (IF) staining of platelet-derived growth factor receptor a (PDGFRa) and vimentin in the old human skin. Dotted lines in IF images mark the epidermal basement membrane. Nuclei are stained with DAPI. (C) Pie chart showing the percentage of vimentin+ cells among PDGFRα⁺ cells in the dermis (n=10 old skin samples, female skin donors, average age: 61.3). (D) Pie chart showing the percentage of PDGFRα⁺ cells among vimentin⁻ cells in the dermis (n=10 old skin samples, female skin donors, average age: 61.3). (E) Percentage of p16^{INK4a}-positive senescent fibroblasts of total fibroblasts per skin sample. Cells are counted blindly and averaged across 10 randomly selected high-power fields (hpf) per skin sample (n=23 in the young and n=31 in the old skin group). (F) Representative IF staining of p21″, p16^{INK4a+}, and vimentin in the old human skin. Nuclei are stained with DAPI. (G) Pie chart showing the percentage of p21⁺ cells in p16^{INK4a+} vimentin⁺ cells and p21⁺ cells in p16^{INK4a}-vimentin⁻ cells in the dermis (n=10 old skin samples, female skin donors, average age: 61.3, chi-squared test). (H) Quantification of p21⁺ fibroblasts in the young and old skin samples (n=10 in the young and n=10 in the old skin group, female skin donors, average age: young: 19.4 and old: 61.3). Cells are counted blindly and averaged across 10 randomly selected hpf per sample. (I) Representative IF staining of SenTraGor (GL13), p16^{INK4a} and vimentin in old human skin samples. Nuclei are stained with DAPI. Bar graphs show mean±SD, Mann-Whitney U test, scale bars, 100 μm.

FIGS. 3A-3J. Epidermal thickness and density of blood/lymphatic vessels are reduced with age, but they do not correlate with the number of senescent dermal cells in the old skin. (A) Representative hematoxylin and eosin (H&E) staining of young and old skin samples. Green brackets highlight epidermal thickness. (B) Quantification of the epidermal thickness (Mann-Whitney U test). (C) Correlation between the epidermal thickness and the number of dermal p16^{INK4a+} cells in the old skin samples (Student's t test for the Pearson correlation coefficient). (D) Representative IF staining of CD31 (a marker for blood endothelial cells) in young and old skin samples. Arrows point to CD31⁺ blood endothelial cells in the dermis. (E) The number of dermal CD31⁺ blood endothelial cells per hpf (Mann-Whitney U test). (F) Correlation between the number of dermal CD31⁺ blood endothelial cells and the number of dermal p16^{INK4a+} cells in the old skin samples (Student's t test for the Pearson correlation coefficient). (G) Representative IF staining of lymphatic vessel endothelial hyaluronan receptor 1 (LYVE-1) (a marker for lymphatic endothelial cells) in young and old skin samples. Arrows point to LYVE-1⁺ lymphatic endothelial cells in the dermis. (H) The number of dermal LYVE-1⁺ lymphatic endothelial cells per hpf (Mann-Whitney U test). (I) Correlation between the number of dermal LYVE-1⁺ lymphatic endothelial cells and the number of dermal p16^{INK4a+} cells in the old skin samples (Student's t test for the Pearson correlation coefficient). (J) Immune cell quantification in the human skin. The number of dermal CD3⁺ cells indicative of T cells; CD3⁺ CD4⁺ cells indicative of CD4⁺ T cells; CD4⁺ T-bet⁺ cells indicative of Th1; CD4⁺ GATA3⁺ cells indicative of Th2 cells; CD4⁺ ROR-γt⁺ cells indicative of Th17 cells; CD4⁺ Foxp3⁺ cells indicative of regulatory T cells (Tregs); CD3⁺ CD8⁺ cells indicative of CD8⁺ T cells; CD3⁺ TCR γδ⁺ cells indicative of γδ T cells; CD3⁺ CD56⁺ cells indicative of NKT cells; CD11c⁺ CD141⁺ cells indicative of cDC1; CD1c⁺ CD11c⁺ CD207 cells indicative of cDC2; CDla⁺ CD207⁺ cells indicative of Langerhans cells (LCs); CD68-CD86⁺ cells indicative of M1 macrophages; CD68⁺ CD206⁺ cells indicative of M2 macrophages; mast cell tryptase*cells indicative of mast cells; neutrophil elastase+ cells indicative of neutrophils; and CD3⁻ CD56^{|} cells indicative of NK cells per hpf. Note that these data are shown as a heatmap in FIG. 4A. Nuclei are stained with DAPI and dotted lines in IF images mark the epidermal basement membrane. Cells are counted blindly and averaged across 10 randomly selected hpf per skin sample. Bar graphs show mean±SD. n=23 in the young and n=31 in the old skin group, *p<0.05. **p<0.01, and ***p<0.0001, ns: not significant, Mann-Whitney U test. Scale bars, 100 μm.

FIGS. 4A-4I. CD4 CTLs are prominent cytotoxic lymphocytes in the human skin, and their number is inversely correlated with the number of dermal senescent cells in the old skin. (A) Heatmap of aging-associated changes in the number of dermal immune cells, determined by multiplex immunostaining on tissue sections. The intensity represents a relative change compared with the young group. n=23 in young and n=31 in the old skin group. (B) Correlation between the number of dermal senescent cells and dermal CD4 CTLs in old skin samples (n=31 old skin samples, Student's t test for the Pearson correlation coefficient). (C) Representative IF staining of CD4 and perforin in young and old human skin samples. Note that CD4⁺ cells are CD3⁺ T cells. (D) Quantification of dermal CD4⁺ perforin⁺ CD4 CTLs per hpf (n=23 in young and n=31 in old skin group, Mann-Whitney U test). (E) Pie chart showing the percentage of perforin⁺ cells in the old dermis (n=31 old skin samples). (F) Volcano plot of RNA-seq data displaying the gene expression pattern in the old versus young human skin samples. Significantly upregulated genes are to the left of 0 on the x-axis, and downregulated genes are to the right of 0 on the x-axis (p<0.05 is considered significant). Selected immune-related genes based on cluster analysis are indicated. (G) Representative IF staining of CXCL9 in the old human skin samples. (H) Quantification of dermal CD4 CTLs per hpf in CXCL9^{high} versus CXCL9^{low} group of old skin samples (n=11 in each group, Mann-Whitney U test). (I) Quantification of p16^{INK4a} positive dermal senescent cells per hpf in CXCL9^{high} versus CXCL9^{low} group of old skin samples. (n=11 in each group, Mann-Whitney U test.) Nuclei are stained with DAPI. Dotted lines in IF images mark the epidermal basement membrane. Cells are counted blindly and averaged across 10 randomly selected hpf per skin sample. Bar graphs show mean±SD. Scale bars, 100 μm.

FIGS. 5A-5C. scRNA-seq analysis of immune cells isolated from old human skin. (A) Exemplary schematic diagram of workflow for scRNA-seq analysis on human skin samples. PI, propidium iodide (marking dead cells). (B) Uniform manifold approximation and projection (UMAP) plot of lymphocyte-enriched CD45 cells sorted from the old human truncal skin samples. A total of 17,624 cells were analyzed from three independent donors (female skin donors, age: 50, 65, and 67 years). (C) Violin plots displaying the distribution of gene expression levels of the T cell-defining markers and select cytotoxic T cell-associated genes in conventional CD4⁺ T cells, CD4 CTLs, and CD8⁺ T cells. DESeq2 was used for DE analysis and p value calculation.

FIGS. 6A-6E. CXCL9 promotes the migration of human skin-resident CD4 CTLs. (A) Representative flow-cytometry plots demonstrating the gating strategy to identify CD4⁺ and CD8⁺ T cells in the human skin. The percent cells in each gate are shown on the flow-cytometry plots. Zombie fixable viability dye (Viability) marks dead cells. (B) Representative flow-cytometry plots of CXCR3 expression on CD4⁺ and CD8⁺ T cells in the human skin. Numbers on the flow-cytometry plots represent the percent cells within each gate. (C) The experimental scheme for trans-well migration assay using the immune cells isolated from the human skin. (D and E) The effect of CXCL9 recombinant protein (585 ng/mL) treatment on the migration of human skin CD4 CTLs (D) and CD8⁺ T cells (E). Skin samples from two independent donors were used to isolate skin-resident immune cells (female skin donors, age: 60 and 73 years). Bar graphs show mean±SD, Mann-Whitney U test.

FIGS. 7A-7K. Skin-resident T cells eliminate senescent fibroblasts in an HLA-II-dependent manner. (A) Representative senescence-associated b-galactosidase (SA-β-Gal or X-gal) staining of normal and senescent dermal fibroblasts. (B) Heatmap representing expressions of immune cell-activating ligands genes in senescent versus normal fibroblasts from RNA-seq data. (D) indicates downregulation (log₂ fold change <−0.4, *p<0.05), and (U) indicates upregulation (log₂ fold change>0.4, *p<0.05) in senescent (n=3) versus normal fibroblasts (n=4). (C and D) Representative flow-cytometry histogram (C) and relative fluorescence intensity (RFI) (D) of ULBP2 on the surface of normal and senescent fibroblasts (n=7 pairs of old human dermal fibroblasts [female skin donors, age range: 43-70, average age: 53.7], paired t test). (E and F) Representative flow-cytometry histogram (E) and RFI (F) of HLA-II on the surface of normal and senescent fibroblasts (n=7 pairs of old human dermal fibroblasts [female skin donors, age range: 43-70, average age: 53.7], paired t test). (G) Representative IF staining of HLA-II, p16^{INK4a}, and vimentin in young and old skin samples. Arrows point to HLA-II^{high} senescent fibroblasts in the dermis. Dotted lines mark the epidermal basement membrane. (H) Quantification of HLA-II^{high} senescent fibroblasts in the skin per hpf (n=23 in young and n=31 in old skin group, Mann-Whitney U test). (I) Exemplary experimental scheme to assay autologous immune cell-elicited cytotoxicity against senescent fibroblasts. (J) Representative immunocytochemistry (ICC) staining of cleaved caspase-3 and vimentin in normal and senescent fibroblasts after co-culture with skin-derived autologous immune cells. Arrows point to cleaved caspase-3⁺ apoptotic fibroblasts. (K) Frequency of cleaved caspase-3⁺ normal and senescent fibroblasts co-cultured with skin-derived autologous immune cells. Fibroblasts were pre-incubated with pan-HLA-DR blocking or isotype control antibodies. Skin immune cell to fibroblast ratio of 50:1 (n=4 female skin donors, age range: 43-65, average age: 48.8, one-way ANOVA). Skin samples used in co-culture experiments were pan-HCMV antigens positive. Nuclei are stained with DAPI. Cells are counted blindly and averaged across 10 randomly selected hpf per sample. Bar graphs show mean±SD. Scale bars, 100 μm.

FIGS. 8A-8M. ULBP2 and HLA-II are expressed on the surface of senescent fibroblasts. (A) The gene set enrichment analysis plot of cellular senescence in replication-induced senescent dermal fibroblasts (n=3) compared with normal dermal fibroblasts (n=4) from RNA-seq data. (B) Representative IF staining of p16^{INK4a}, ULBP2, and vimentin in the old skin. The arrows point to a ULBP2⁺ senescent fibroblast in the dermis. Nuclei are stained with DAPI, and dotted lines mark the epidermal basement membrane. (C) Quantification of ULBP2+p16^{INK4a+} vimentin⁻ senescent fibroblasts in the young and old skin samples (n=15 in the young and n=15 in the old skin group, female skin donors, average age: young: 20.7 and old: 62.3). Cells are counted blindly and averaged across 10 randomly selected hpf per sample (Mann-Whitney U test). (D) Quantification of dermal CD4 CTLs per hpf in HLA-II^{high} positive (HLA-II^{high} senescent fibroblasts/hpfR0.2) versus HLA-II^{high} negative (HLA-II^{high} senescent fibroblasts/hpf=0) skin samples (n=22 in HLA-II^{high} positive and n=19 in HLA-II^{high} negative group across young and old skin samples, Mann-Whitney U test). (E) Representative X-gal staining of human dermal fibroblasts radiated with 5 J of UVA versus sham. (F and G) Representative flow-cytometry histogram (F) and relative fluorescence intensity (RFI) (G) of ULBP2 on the surface of dermal fibroblasts 1 day after radiation with 5 J of UVA versus sham. (H and I) Representative flow-cytometry histogram (H) and RFI (I) of HLA-II on the surface of dermal fibroblasts 1 day after radiation with 5 J of UVA versus sham. (J and K) Representative flow-cytometry histogram (J) and RFI (K) of ULBP2 on the surface of dermal fibroblasts 1 day after radiation with 10 J of UVA versus sham. (L and M) Representative flow-cytometry histogram (L) and RFI (M) of HLA-II on the surface of dermal fibroblasts 1 day after radiation with 10 J of UVA versus sham. n=7 pairs of adult human dermal fibroblasts, female skin donors, age range: 43-70, average age: 53.7, paired t test. Scale bars, 100 μm.

FIGS. 9A-9L. Allogeneic skin-resident T cells selectively eliminate senescent fibroblasts in an HLA-II-dependent manner. (A) Representative ICC staining of cleaved caspase-3 and vimentin in the normal and senescent human dermal fibroblasts after co-culture with skin-derived allogeneic immune cells. Nuclei are stained with DAPI. Arrow points to a cleaved caspase-3⁺ apoptotic fibroblast. (B) Frequency of cleaved caspase-3⁺ normal and senescent fibroblasts after co-culture with skin-derived allogeneic immune cells for 6 h, using skin immune cell to fibroblast ratios of 0:1, 10:1, and 50:1 (n=5 female skin donors, average age: 50.4, Mann-Whitney U test). (C) Frequency of cleaved caspase-3⁺ normal or senescent fibroblasts after co-culture with skin-derived allogeneic immune cells for 6 h. Fibroblasts were preincubated with pan-HLA-DR blocking or isotype control antibody for 18 h. Skin-derived immune cell to fibroblast ratio of 50:1 (n=3 female skin donors, average age: 50.3, one-way ANOVA). Cells are counted blindly and averaged across 10 randomly selected hpf per sample. (D) RFI of HLA-II on the surface of senescent fibroblasts that were transfected with control versus CIITA siRNA for 48 h (Mann-Whitney U test). (E) Representative ICC staining of cleaved caspase-3 and vimentin in senescent fibroblasts after co-culture with skin-derived immune cells. Nuclei are stained with DAPI. (F) Frequency of cleaved caspase-3⁺ normal and senescent fibroblasts co-cultured with skin-derived immune cells. Fibroblasts were transfected with control versus CIITA siRNA for 48 h prior to co-culture with immune cells. Skin immune cell to fibroblast ratio of 50:1 (one-way ANOVA). Skin samples from two independent donors were used to isolate skin-resident immune cells (female skin donors, age: 60 and 73 years). (G and H) Representative flow-cytometry plots (G) and quantification (H) of CD107a′ cells among skin-resident (CD69+) CD4⁺ T cells following co-culture with normal versus senescent fibroblasts. The percentage of CD107a⁺ CD4⁺ T cells in the gate is shown on the flow-cytometry plots. (I and J) Representative flow-cytometry plots (I) and quantification (J) of CD137⁺ cells among skin-resident CD4⁺ T cells following co-culture with normal versus senescent fibroblasts. The percentage of CD137⁺ CD4⁺ T cells in the gate is shown on the flow-cytometry plots. (K and L) Representative flow-cytometry plots (K) and quantification (L) of IFNγ⁺ cells among skin-resident CD4⁺ T cells following co-culture with normal versus senescent fibroblasts. The percentage of IFNγ⁺ CD4⁺ T cells in the gate is shown on the flow-cytometry plots. Skin samples from two independent donors were used to isolate skin-resident immune cells (female skin donors, age: 60 and 73 years). Bar graphs show mean±SD, Mann-Whitney U test, scale bars, 100 μm.

FIGS. 10A-10H. HCMV DNA and RNA are upregulated in the old human skin, (A) Quantitative PCR for HCMV DNA detection in HCMV-versus sham-infected human fetal fibroblasts using HCMV UL83 primers. Data are presented as the ratio of GAPDH expression (n=4 per group). (B) Quantitative PCR for HCMV DNA detection in the young versus old human skin samples using HCMV UL83 primers. Data are presented as the ratio of GAPDH expression (n=21 in the young and n=30 in the old skin group, female skin donors, average age: young: 23.3 and old: 62.5). (C) Quantification of HCMV RNA in situ hybridization (ISH) count in the young versus old skin samples. HCMV RNA ISH positive signals are counted blindly and averaged across three hpf with HCMV RNA ISH signal if any were detected per skin sample (n=21 in the young and n=30 in the old skin group, female skin donors, average age: young: 23.3 and old: 62.5). (D) Representative images of HCMV RNA ISH (arrow) immunohistochemistry staining and IF staining with vimentin in the dermis of young and old skin (scale bars, 100 μm). (E) Representative images of HCMV RNA ISH with IF staining of vimentin in dermal fibroblasts in the young and old skin. Arrows point to HCMV RNA⁺ signals in fibroblasts (scale bar, 10 μm). HCMV RNA ISH probe is designed to detect UL123 (IE1) transcript. Nuclei are stained with DAPI. (F) Representative IF staining of pan-HCMV antigens (Ag) and vimentin in old human skin (scale bar, 100 μm). (G) Pie chart showing the percentage of young and old human skin samples with pan-HCMV Ag′ cells in the dermis (n=23 in the young and n=31 in the old skin group, chi-squared test). (H) HCMV DNA (UL83) expression in the old skin samples that have pan-HCMV Ag⁺ cells (pan-HCMV Ag-positive, n=21) versus old skin samples that do not have detectable pan-HCMV Ag⁺ cells (pan-HCMV Ag-negative, n=10). Bar graphs show mean±SD, Mann-Whitney U test.

FIGS. 11A-11G. Senescent fibroblasts express HCMV-gB antigen. (A) Representative IF staining of p16^{INK4a}, HCMV-gB, and vimentin in the old human skin. Arrows point to HCMV-gB⁺ senescent fibroblasts. Dotted lines mark the epidermal basement membrane. (B) Quantification of HCMV-gB⁺ p16^{INK4a+} fibroblasts in dermis per hpf (n=23 in young and n=31 in old skin group, Mann-Whitney U test). (C) Representative ICC staining of HCMV-gB and vimentin in normal and senescent fibroblasts. Arrows point to HCMV-gB⁺ fibroblasts. (D) Frequency of HCMV-gB⁺ normal versus senescent fibroblasts. (E) Quantitative PCR for HCMV DNA detection in normal and replication-induced senescent fibroblasts using HCMV UL83 primer. Data are presented as the ratio of GAPDH expression. (F) Frequency of HCMV-gB⁺ fibroblasts 5 days after 5 J UVA versus sham UVA radiation. (G) Quantitative PCR for HCMV DNA detection in fibroblasts 1 day after 5 J UVA versus sham UVA radiation using HCMV UL83 primer. Data are presented as the ratio of GAPDH expression. Nuclei are stained with DAPI. Cells are counted blindly and averaged across 10 randomly selected hpf per sample. The bar graph shows mean±SD. Skin samples used for fibroblast isolation were pan-HCMV antigens positive. n=7 pairs of adult human dermal fibroblasts (female skin donors, age range: 43-70, average age: 53.7, paired t test). Scale bars, 100 μm.

FIGS. 12A-12B. HCMV-IE1/IE2 detection in the old human skin. (A) Representative IF staining of HCMV-IE1/IE2 (Ag) and vimentin in young and old human skin samples. Nuclei are stained with DAPI. (B) Pie charts showing the percentage of the skin samples with HCMV-IE1/IE2⁺ cells in the dermis. n=23 in the young and n=31 in the old skin group, chi-squared test, scale bar, 100 μm.

FIGS. 13A-13C. HCMV-gB is localized to early endosomes in senescent fibroblasts. (A) Representative ICC staining of HCMV-gB, Rab5, and vimentin in HCMV-versus sham-infected human fetal fibroblasts. Arrows point to HCMV-gB and Rab5 signal co-localization in fibroblasts. (B) Representative ICC staining of HCMV-gB, Rab5, and vimentin in normal and replication-induced senescent fibroblasts. Arrows point to HCMV-gB and Rab5 signal co-localization in a fibroblast. (C) Representative ICC staining of HCMV-gB, Rab5, and vimentin in fibroblasts 5 days after 5 J UVA or sham UVA radiation. Arrows point to HCMV-gB and Rab5 signal co-localization in a fibroblast. Nuclei are stained with DAPI; scale bars, 100 μm.

FIGS. 14A-14H. HCMV-gB antigen activates skin-resident CD4 CTLs. (A) Pie charts showing the gene usage of TRBV06-05-expressing T cell clones in six HLA-DRB1*07: 01⁺ old skin samples (female skin donors, age range: 59-74, average age: 63.3). (B) Representative flow-cytometry plots of HCMV-gB-tetramer⁺ perforin⁺ CD4⁺ T cells among skin-resident CD4⁺ T cells stained with HCMV-gB tetramer versus control tetramer. Skin-resident CD8⁺ T cells stained with HCMV-gB tetramer are shown as a negative control. The percentage of tetramer⁺ perforin⁺ cells is shown in the gate on the flow-cytometry plots. (C) Quantification of HCMV-gB⁺ perforin⁺ CD4⁺ T cells as a percent of total skin-resident CD4⁺ T cells (n=9 skin samples per group, female skin donors, average age: young: 19.3 and old: 63.3, Mann-Whitney U test). (D) Representative ICC staining of HCMV-gB, cleaved caspase-3, and vimentin in normal and senescent fibroblasts after co-culture with skin-derived autologous immune cells. Arrows point to an HCMV-gB⁺ apoptotic fibroblast. (E and F) Frequency of cleaved caspase-3+HCMV-gB⁺ (E) and cleaved caspase-3-HCMV-gB⁺ (F) normal and senescent fibroblasts co-cultured with skin-derived autologous immune cells. Fibroblasts were pre-incubated with pan-HLA-DR blocking or isotype control antibodies. Skin immune cell to fibroblast ratio of 50:1 (n=4 individual donors, female skin donors, age range: 43-65, average age: 48.8, one-way ANOVA). (G and H) Representative flow-cytometry plots (G) and quantification (H) of skin-resident IFNγ⁺ perforin⁺ CD4⁺ T cells following stimulation with recombinant HCMV-gB, HCMV-gH and/or poly (I:C). The percentage of CD4⁺ T cells in each quadrant is shown on the flow-cytometry plots. Stimulation with phorbol-12-myristate-13-acetate and ionomycin (PMA/ion) was used as a positive control (n=18 in the negative control, n=18 in poly (I: C), n=18 in HCMV-gB, n=18 in HCMV-gB+poly (I: C), n=16 in PMA/ionomycin, n=12 in HCMV-gH, and n=12 in HCMV-gH+poly (I: C) group, one-way ANOVA, Dunnett's multiple comparison test). Skin samples from five independent donors were used to isolate skin-resident immune cells (female skin donors, age range: 48-73, average age: 57.6). Nuclei are stained with DAPI. Cells are counted blindly and averaged across 10 randomly selected hpf per sample. Skin samples used for fibroblast isolation were pan-HCMV antigens positive. Bar graphs show mean±SD. Scale bar, 100 μm.

FIGS. 15A-15B. Butyrate and pentanoate boost CD4 CTL-mediated clearance of senescent cells. (A) The frequency of cleaved caspase-3⁺ senescent fibroblasts after co-culture for 6 h with skin T cells, which are preincubated with each 5 mM SCFAs for 2 days, using a skin T cell to fibroblast ratio of 50:1. All data are expressed as the mean±SD. n=3 individuals. (B) The effect of each SCFA (5 mM) treatment on the cytotoxicity of CD4⁺ T cells. Flow cytometry analysis of perforin and CD107a expression on CD4⁺ T cells exposed to each SCFA for 24 h. n=2 individuals. All data are expressed as the mean±SD.

### DETAILED DESCRIPTION

Accumulating evidence demonstrates that genetic or pharmacological approaches to eliminate senescent cells from aging tissues can restore tissue homeostasis and lead to increased healthy lifespan in mice^{3,5}. However, current genetic and pharmacological approaches produce substantial side effects and lack long-term durability^{1,4}. Considering that the senescent cells produce SASP, their immunogenic phenotype marks them as potential targets for surveillance and clearance by the immune system. However, the immune clearance of senescent cells is hampered by the immunomodulatory molecules expressed by the senescent cells and the immunosuppressive factors in their microenvironment in the experimental mouse models⁶⁻⁹. More importantly, it remains unclear how immunity against senescent cells is regulated in humans.

The development and phenotype of senescent cells fundamentally differ between mice and humans including the role of telomere shortening and oxidative stress in the induction of cellular senescence¹⁰⁻¹⁴. A notable limitation of experimental mouse models to study senescence relates to their inability to fully capture the spectrum of immune responses against senescent cells due to their lack of pathogen exposure and evolutionarily distinct immunosurveillance mechanisms¹⁵⁻¹⁷. Therefore, the mechanism of senescent cell clearance in humans was explored and a novel commensal virome-immune axis that prevents the accumulation of senescent cells in aging skin was discovered.

The present findings reveal a previously unknown role for CD4 CTLs in the clearance of senescent cells that experience cytomegalovirus reactivation. These findings have significant implications for understanding how the immunity against senescent cells is regulated in humans. Senescent cells, mainly dermal fibroblasts, accumulate in the old compared with young skin; however, their number does not linearly correlate with advance age in the elderly. Among the possible explanations for the age-independent accumulation of senescent cells, CD4 CTLs were identified as critical regulators of senescent cells in old skin. CD4 CTLs recognize and eliminate senescent fibroblasts at least in part by targeting the HCMV-gB antigen, which highlights a commensal-like function for HCMV in the immunosurveillance of aging cells in immunocompetent hosts.

Cytotoxic lymphocytes utilize the perforin/granzyme pathway to kill virus-infected and tumor cells.⁴⁰ Unexpectedly, it was found that CD4⁺ T cells are a prominent population of perforin-expressing cytotoxic lymphocytes in old skin by histological analysis, and their frequency in the dermis negatively correlates with the accumulation of senescent cells in old human skin. Increased CXCL9 chemokine expression by aging keratinocytes provides an explanation for the higher frequency of dermal CD4 CTLs in old skin, suggesting that the chemokine milieu of the skin is a major determinant of CD4 CTL infiltration in the old skin.

As demonstrated herein, replication and UVA-induced senescent human fibroblasts upregulate HLA-II and HCMV-gB expression. Endogenous HCMV-derived gB is sorted into endosomes and presented on HLA-II to CD⁴⁺ T cells.41 Accordingly, CD4 CTLs from the human skin specifically eliminated HCMV-gB⁺ senescent fibroblasts in an HLA-II-dependent manner.

CD4 CTLs can be detected during viral infection in humans, where their direct anti-viral effector function helps control the infection^{27 28}. Notably, the high presence of circulating CD4 CTLs is a characteristic of supercentenarians, who live more than 110 years in good health due to the delayed onset of age-related diseases and reduced morbidity²⁹. Without wishing to be bound by theory, in addition to fighting infection, CD4 CTLs contribute to the elimination of senescent cells and their associated diseases in supercentenarians to achieve exceptional longevity.

Although HCMV is an infectious cause of birth defects and can cause serious morbidity in severely immunocompromised individuals,^{42,43} it is a pervasive herpesvirus that establishes lifelong latent infection in the majority of the human population without any symptoms in immunocompetent hosts.⁴⁴ HCMV produces several immunodominant antigens and profoundly influences the repertoire of adaptive immunity in healthy individuals during aging.⁴⁵ HCMV-specific CD4 CTLs display high cytotoxicity while producing a low amount of cytokines, enabling them to effectively fight HCMV reactivation while minimizing tissue inflammation³⁰. Although HCMV is known to establish latency in myeloid cells, fibroblasts are found to be predominant in the pool of HCMV-infected cells in vivo.^{46, 47} Accordingly, HCMV is detected in dermal fibroblasts in normal human skin.

As shown herein, HCMV is reactivated upon the induction of cellular senescence. Without wishing to be bound by theory, this could explain how CD4 CTLs, and likely CD8 CTLs and NK cells, can detect the senescent fibroblasts in the mix of healthy cells, which are also infected with HCMV as a commensal virus. This finding is supported by the observation that HCMV upregulates p16^{INK4a} to increase its replication³¹. HCMV reactivation upon replication- and UVA-induced senescence, coupled with HLA-II and stress ligands induction on the senescent cells, enables the HCMV-specific CD4 CTLs to target and eliminate the senescent cells effectively. Other innate and adaptive immune cell types, including macrophages, NK cells, and CD8⁺ T cells, also play pivotal roles in the clearance of senescent cells.^{48,49,50-53} Considering that senescent fibroblasts highly express ULBP2, a ligand for activating NKG2D receptor expressed broadly on cytotoxic lymphocytes, innate and adaptive immune cells likely participate in the clearance of senescent cells together with CD4 CTLs in the skin.

The expression of HCMV-gB in the senescent fibroblasts in the presence of low immediate-early 1 (IE1)/immediate-early 2 (IE2) expression and the absence of lytic infection may represent an interesting biology associated with an abortive replication cycle. For example, senescent cells may persist in the face of HCMV reactivation and not enter a lytic phase even though late viral proteins are expressed, which in turn could inhibit lytic viral spread. HCMV-infected adult fibroblasts upregulate HLA-II during cellular senescence similar to uninfected embryonic fibroblasts, suggesting that cellular senescence can overcome the suppression of HLA-II by HCMV, which undergoes reactivation in senescent cells. Interestingly, previous reports have shown that herpesviruses preferentially downregulate HLA-I expression rather than HLA-II, which leads to a prominent role for CD4 CTLs in the clearance of virus-infected cells.^{54,55} Thus, cellular senescence may function as a natural antiviral defense mechanism by inhibiting lytic viral spread while creating an immunogenic target for clearance.^{56,57} This protective function of cellular senescence as an antiviral machinery highlights a notable aspect of immune-virome interactions, which promotes the homeostasis of virus-colonized organs.

The present findings provide a novel insight into the complex human immune system-commensal virome interactome. Without wishing to be bound by theory, these results indicate that the human immune system has evolved to establish a symbiotic relationship with HCMV that prevents the accumulation of senescent cells during aging. Thus, HCMV can have a beneficial impact on human health, which is mediated by a competent anti-viral T cell immunity. Described herein are methods that include vaccination against commensal HCMV antigens to boost anti-HCMV T cell immunity and reduce the risk of, delay the onset of, and/or slow the progression of aging and aging-associated diseases; without wishing to be bound by theory, it is believed that the vaccines increase senescent cell clearance.

### Methods of Inducing Immunity

The methods described herein include methods of treating, or reducing the risk of developing, an aging-associated disease or condition. Also described herein are methods of delaying the onset or slowing progression of aging in a subject as well as methods of reducing the level of senescent fibroblasts in a subject. The methods include administering one or more doses of the vaccine compositions described herein to a subject, e.g., a subject in need thereof. The vaccines induce T cell immunity against commensal viruses that have already infected the tissue, with the goal not to prevent or eliminate the infection but rather to use the virus presence in all cells to boost the detection of senescent cells and their elimination by T cells.

The compositions are administered in an effective amount. An “effective amount” is an amount sufficient to effect beneficial or desired results. For example, an effective amount is one that achieves a desired therapeutic effect, e.g., an amount necessary to treat a disease, or to reduce risk of development of disease or disease symptoms (also referred to as a therapeutically effective amount or a prophylactically effective amount, respectively). An effective amount can be administered in one or more administrations, applications, or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments. For example, the methods can include administering a first dose, followed by a second dose at a later time (e.g., a “booster” dose), e.g., at 1, 2, 4, 6, 8, 12, 18, 24, or 52 weeks later.

Dosage, toxicity, and therapeutic efficacy of the therapeutic compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit high therapeutic indices are preferred. While compositions that exhibit toxic side effects may be used, care should be taken to minimize and reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compositions used in the methods described herein, the therapeutically effective dose can be estimated initially from cell culture assays. A dose may be formulated in animal models. Such information can be used to determine useful doses more accurately in humans.

Many conditions and diseases are associated with aging. For example, Aging is considered a risk factor for cancer and other diseases. See, e.g., Laconi, et al. Br J Cancer. 2020 March; 122 (7): 943-952. Aging-associated conditions include graying hair, hearing loss, a cataract, frailty, and sarcopenia. Aging-associated diseases can include cancers, cardiovascular diseases, neurodegenerative diseases, renal diseases (e.g., chronic kidney disease), autoimmune diseases, arthritis (e.g., osteoarthritis), osteoporosis, macular degeneration, chronic obstructive pulmonary disease (COPD), glaucoma, obesity, fibrosis (e.g., liver fibrosis), cirrhosis (e.g., liver cirrhosis), hepatic steatosis, and diabetes. Non-limiting examples of such cancers include osteosarcoma, breast cancer, prostate cancer, colorectal cancer, lung cancer, melanoma, kidney cancer, lymphoma, uterine cancer, pancreatic cancer, non-melanoma skin cancer, and bladder cancer. Non-limiting examples of a neurodegenerative disease include dementia, ataxia, Huntington's disease, a motor neuron disease, or a tau-mediated neurodegenerative disease (e.g., Alzheimer's disease, Parkinson's disease, or progressive supranuclear palsy). Non-limiting examples of a cardiovascular disease include atherosclerosis, idiopathic pulmonary fibrosis, coronary heart disease, congestive heart failure, coronary artery disease, peripheral arterial disease, valvular heart disease, arrhythmias (e.g., arterial fibrillation), ischemic cardiomyopathy, hypertension, and stroke. Non-limiting examples of an autoimmune disease include multiple sclerosis, Crohn's disease, rheumatoid arthritis, an antineutrophilic cytoplasmic antibody (ANCA) associated vasculitide, and systemic lupus erythematosus. Non-limiting examples of fibrosis include systemic sclerosis, scleroderma, idiopathic pulmonary fibrosis, and interstitial lung disease.

The methods can also include any appropriate method of measuring the onset or progression of any of the diseases described herein. For example, any of the methods described herein can include any appropriate method of measuring the onset or progression of cancer, fibrosis, cardiovascular diseases, and neurodegenerative diseases.

The methods can also include administration of one or more other treatments known in the art for treating an aging-associated disease or condition, e.g., in subjects who have an aging-associated disease or condition, or treatment to reduce the risk of developing an aging-associated disease or condition. For example, a combination treatment with the compositions described herein plus a standard treatment for any of the conditions or diseases described herein can be used in combination with the present methods. In some embodiments, these agents boost antigen presentation (innate signals) while the present compositions boost antigen recognition by T cells.

### Commensal HCMV Vaccines

Also described herein are compositions that can be used to induce a T cell-based immune response against cytomegaloviruses, thereby treating, or reducing the risk of developing, an aging-associated disease or condition. A number of CMV vaccines have been developed including live attenuated, plasmid DNA, viral-vectored, and subunit vaccines (see, e.g., Rieder and Steininger, Clin Microbiol Infect 2014; 20(Suppl. 5):95-102; McVoy Clin Infect Dis 2013; 57(Suppl.4):S196-9). In some embodiments, the present compositions can include a live CMV vaccine or a live attenuated CMV vaccine. Many CMV vaccines include CMV glycoprotein B (gB) or antigenic portions thereof, delivered either as a protein or as a nucleic acid encoding the protein (e.g., plasmid DNA or mRNA). Non-limiting examples of CMV vaccines include CMVPepVax, Chiron gB, the disabled infectious single cycle (DISC) V160 vaccine, a modified vaccinia virus Ankara (MVA) vaccine vector to express glycoprotein B, phosphoprotein 65, and all five subunits of the pentamer complex, and an mRNA platform encoding gB, pp65, IE1, or pentameric complex. See, e.g., Cui and Snapper, Hum Vaccin Immunother. 2019; 15(11): 2673-2683.

In some embodiments, the present compositions can include a plurality of proteins, e.g., virus-like particles containing glycoprotein B from commensal human cytomegaloviruses, e.g., CMV strains such as AD169, Towne, Toledo, PH, TR, FIX, VR1814, Merlin, and TB40/E. See, e.g., Murphy et al. Proc Natl Acad Sci USA. 2003 Dec. 9; 100(25): 14976-81; and Wilkinson et al. Med Microbiol Immunol. 2015; 204(3): 273-284. Non-limiting examples of an antigenic portion of CMV glycoprotein B include antigenic domain (AD) 1, AD-2, AD-3, AD-4, AD-5, and AD-6. For example, AD-1 includes approximately 80 amino acids between positions 560 and 640 of glycoprotein B (e.g., of HCMV strain AD169). In some embodiments, AD-2 includes at least two distinct sites between amino acids 50 and 77 of glycoprotein B. AD-3 includes intraluminal/intraviral portions of glycoprotein B. AD-4 includes a site between amino acids 133-343 of glycoprotein B. AD-5 includes a discontinuous domain from amino acids 121-132 and 344-438. AD-6 includes amino acids 648-697 of glycoprotein B. See, e.g., Pötzsch, et al. PLOS Pathog. 2011 August; 7(8): e1002172 and Gomes et al. at Commun. 2023 Feb. 23; 14(1): 1041. Non-limiting examples of CMV vaccines that include CMV glycoprotein B or antigenic portions thereof include vaccines comprising a soluble, recombinant glycoprotein B that is a truncated version of glycoprotein B that lacks the transmembrane domain (e.g., Chiron gB), and a vaccine comprising a recombinant trimeric glycoprotein B. See, e.g., Cui and Snapper, Hum Vaccin Immunother. 2019; 15(11): 2673-2683.

In some embodiments, the present compositions include a plurality of antigenic peptides derived from (i.e., comprising a fragment of, i.e., consecutive amino acids from) proteins, e.g., glycoprotein B, from commensal human cytomegaloviruses. The peptides can be derived from any antigenic protein in the virus: in some embodiments, the peptides are derived from glycoprotein B (e.g., a glycoprotein antigenic domain described herein). Sequences for CMV glycoprotein B include APB97351.1; ABQ23592.1; QTT59567.1; QTT59229.1; QTT59398.1; QTTS9064.1; and APA45814.1. In some embodiments, at least 50 or more, 100, 150, 200, 250, 300, 350, 400, 450, 500, or more different peptides (i.e., peptides having different sequences) are included in the compositions. In some embodiments, at least 50 or more, 100, 150, 200, 250, 300, 350, 400, 450, 500, or more different peptides from each virus strain are included in the compositions, and peptide from two or more virus strains are included.

In some embodiments, the peptides are of a length that is optimized for MHCI/MHCII presentation, e.g., 9-30 amino acids, e.g., 12-25, 12-18, 12-16, 13-16, 14-16, or 15 amino acids. The sequences of the peptides can be synthetic long overlapping peptides, e.g., identified, e.g., bioinformatically to predict antigenicity and/or generated using a moving window of overlapping peptides to cover the entire protein, e.g., 15 amino acid peptides with 10 amino acid overlap. In some embodiments, overlapping synthetic long peptides (SLPs) are used (Zom et al., Cancer Immunol Res. 2014 August; 2(8): 756-64). The compositions can include a plurality of peptides derived from one or more (e.g., a plurality of) different virus strains. The peptides are preferably synthetic peptides; methods for synthesizing peptides are known in the art, including solution-phase techniques and solid-phase peptide synthesis (SPPS). See, e.g., Petrou and Sarigiannis, Ch. 1-Peptide synthesis: Methods, trends, and challenges, In: Editor(s): Sotirios Koutsopoulos, Peptide Applications in Biomedicine, Biotechnology and Bioengineering, Woodhead Publishing, 2018, pages 1-21; and Chandrudu et al., Molecules 2013, 18, 4373-4388.

In some embodiments, the present compositions can include a plurality of DNA plasmids and/or RNA replicons that contain nucleotide sequences to express proteins or antigenic peptides derived from (i.e., comprising a fragment of, i.e., consecutive amino acids from) proteins, e.g., glycoprotein B, from commensal human cytomegaloviruses, e.g., CMV strains such as AD169, Towne, Toledo, PH, TR, FIX, VR1814, Merlin, and TB40/E.

In some embodiments, the present compositions can include a plurality of viral vectors that are engineered to express proteins or antigenic peptides derived from (i.e., comprising a fragment of, i.e., consecutive amino acids from) proteins, e.g., glycoprotein B, from commensal human cytomegaloviruses, e.g., CMV strains such as AD169, Towne, Toledo, PH, TR, FIX, VR1814, Merlin, and TB40/E. Viral vectors for use in the present methods and compositions include recombinant retroviruses, adenovirus, adeno-associated virus, alphavirus, and lentivirus.

### T Cell Adjuvant

The compositions can also include an adjuvant to increase T cell response. For example, such adjuvants can include short chain fatty acids. Non-limiting examples of short chain fatty acids include acetate, propionate, butyrate, valerate, formate, isobutyrate, isovalerate, and 2-methylbutanoate.

In some embodiments, an oil-in-water emulsion can be included, e.g., an oil-in-water emulsion containing squalene (4.3%) in citric acid buffer with stabilizing nonionic surfactants Tween 80 (0.5%) and Span 85 (0.5%) (e.g., MF59®). In some embodiments, nanoparticles that enhance T cell response can be included, e.g., as described in Stano et al., Vaccine (2012) 30:7541-6 and Swaminathan et al., Vaccine (2016) 34:110-9. See also Panagioti et al., Front. Immunol., 16 Feb. 2018; doi.org/10.3389/fimmu.2018.00276. Alternatively or in addition, an adjuvant comprising a Toll-like receptor agonist (e.g., a Toll-like receptor 9 agonist such as PF03512676), poly-ICLC (carboxymethylcellulose, polyinosinic-polycytidylic acid, and poly-L-lysine double-stranded RNA), Imiquimod, Resiquimod (R-848), CpG oligodeoxynuceotides and formulations (IC31, QB10), AS04 (aluminum salt formulated with 3-O-desacyl-4′-monophosphoryl lipid A (MPL)), AS01 (MPL and the saponin QS-21), MPLA, STING agonists, other TLR agonists, GM-CSF, Fms-like tyrosine kinase-3 ligand (Flt3L), and/or IFA (Incomplete Freund's adjuvant) can also be used. See, e.g., Khong and Willem, Journal for ImmunoTherapy of Cancer 4:56 (2016); Coffman et al., Immunity. 2010 Oct. 29; 33 (4): 492-503; Martins et al., EBioMedicine 3:67-78, 2016; and Del Giudice, Seminars in Immunology, 2018, doi.org/10.1016/j.smim.2018.05.001.

### Compositions

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, intratumoral, intramuscular or subcutaneous administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, intramuscular, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, using a coating such as lecithin, by the maintenance of the required particle size in the case of dispersion and using surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### Subjects

The vaccine compositions described herein can be used to boost immunity against senescent cells in subjects. In some embodiments, the subjects do not have an aging-associated disease (e.g., do not have any of the cancers described herein). In some embodiments, the subjects are at high risk (i.e., have a risk that is above that of the general population) of developing an aging-associated disease. For example, the subject may be an adult. In some embodiments, the subject is 40 years old or older. For example, the subject can be 45, 50, 55, 60, 65, 70, 75, 80, or 85 years old or older.

In some embodiments, the subjects are not infants, e.g., the subjects are not 1 years old or younger. In some embodiments, the subjects do not have congenital CMV (cCMV) disease. In some embodiments, the subjects are not pregnant. In some embodiments, the subjects are not at risk of transmitting a cytomegalovirus to a fetus.

In some embodiments, the subjects have an aging-associated disease (e.g., any of the aging-associated diseases described herein). In some embodiments, the subjects have one or more of a cancer (e.g., any of the cancers described herein), a cardiovascular disease (e.g., any of the cardiovascular diseases described herein), a neurodegenerative disease (e.g., any of the neurodegenerative diseases described herein), a renal disease (e.g., any of the a renal diseases described herein), an autoimmune disease (e.g., any of the autoimmune diseases described herein), arthritis (e.g., osteoarthritis), osteoporosis, macular degeneration, chronic obstructive pulmonary disease (COPD), glaucoma, obesity, fibrosis (e.g., liver fibrosis), cirrhosis (e.g., liver cirrhosis), hepatic steatosis, and diabetes. In some embodiments, the subjects have a cardiovascular disease (e.g., any of the cancers described herein).

In some embodiments, the subjects are not a recipient of a transplanted organ or hematopoietic stem cells. In some embodiments, the subjects are not immunocompromised.

Subjects who can be treated using the present methods include mammals, e.g., human, and non-human veterinary subjects.

### EXAMPLES

The invention is further described in the following examples, which do not limit the scope of the invention described in the claims.

### Methods

The following materials and methods were used in this study.

### Isolation and Culture of Human Dermal Fibroblasts

Dermal fibroblasts were isolated from discarded normal skin samples, which were generated as part of the surgery. Subcutaneous fat tissue was removed from human skin tissue, and the tissue pieces were incubated in dispase solution (Stemcell, Vancouver, Canada, 07913) overnight at 4° C. After digestion, the epidermis was separated from the dermis. The obtained dermis was incubated in collagenase/hyaluronidase (Stemcell, 07912) overnight at 37° C. Fibroblasts were collected through a 70 μm cell strainer and were seeded at a density of 3-5×10⁴ cells/cm² into 75 cm² cell culture flasks, and cultured in DMEM medium (Thermo Fisher Scientific, Waltham, MA 11-965-118), including 10% fetal bovine serum (FBS), 1% penicillin/streptomycin, and 1% glutamine, at 37° C. under an atmosphere of 5% CO₂ in the air. Human fetal dermal fibroblasts (ScienCell Research Laboratories, Carlsbad, CA, 2300) and neonatal dermal fibroblasts (Lonza, Basal, Switzerland, CC-2509) were purchased. Cells were cultured as described above.

### Human Skin Immune Cell Isolation

Immune cells were isolated from human skin as previously described³². Briefly, discarded normal skin samples generated as part of surgery were obtained. Subcutaneous fat tissue was removed from human skin tissue, and the remaining tissue was minced. Skin tissues were minced and digested in RPMI 1640 medium (Thermo Fisher Scientific, 21-870-092) including 0.05% DNase-I (Sigma-Aldrich, St. Louis, MO, 10104159001) and 0.2% collagenase-I (Thermo Fisher Scientific, LS004196) for 2 h at 37° C. Then cells were collected through a 70 μm cell strainer and were incubated in RPMI 1640 medium including 20% FBS, 1% penicillin/streptomycin, 1% glutamine, 0.00035% β-mercaptoethanol, and 2 ng/ml human interleukin (IL)-2 recombinant (BioLegend, San Diego, CA, 589104).

### Histology

Human skin samples were fixed with 4% paraformaldehyde (PFA) and embedded in paraffin. 5 μm sections were cut and deparaffinized. After being permeabilized with 0.2% Triton-X (Thermo Fisher Scientific, BP151) in phosphate-buffered saline (PBS), antigen retrieval was performed using a pressure cooker in antigen unmasking solution (Vector Laboratories, Burlingame, CA, H-3300-250) for 20 min. Slides were washed three times for 5 min each in PBS including 0.1% Tween 20 (Sigma-Aldrich, P1379). Slides were blocked with 5% normal goat serum (Sigma-Aldrich, G9023) and 5% bovine serum albumin (Thermo Fisher Scientific, BP1600) in PBS for 1 h. Slides were stained overnight at 4° C. with primary antibodies (Table 3A) diluted in the blocking buffer. Following primary antibody application, slides were washed and incubated in secondary antibodies (Table 3A) diluted in the blocking buffer for 2 h at room temperature. Slides were washed and stained with 4′,6-diamidino-2-phenylindole (DAPI, Invitrogen, D3571, 1:4000) in PBS for 10 min at room temperature. Slides were washed and mounted with ProLong Gold Antifade Reagent (Thermo Fisher Scientific, P36930). The stained tissues were imaged with a ZEISS confocal microscope (Zeiss, Oberkochen, Germany). Manual counting was performed using the ZEN Blue Software (Zeiss). Cell counts were reported as the average number of cells across 10 randomly selected high power fields (hpf, 200× magnification) per skin sample in each group. For hematoxylin and eosin staining, slides were stained according to standard procedures and mounted with Cytoseal XYL (Thermo Fisher Scientific, 8312-4). Whole-slide imaging was performed using a Zeiss Axio Scan.Z1 (Zeiss).

### Immunocytochemistry

Cells were cultured on chamber slide glasses (CELLTREAT Scientific Products, Pepperell, MA, 229168) and fixed with 4% PFA in 10 min at room temperature and were permeabilized with 0.2% Triton-X in PBS in 10 min at room temperature. Slides were washed three times for 5 min each in PBS. Slides were blocked with 5% normal goat serum and 5% bovine serum albumin in PBS for 30 min. Slides were stained overnight at 4° C. with primary antibodies (Table 3A) diluted in the blocking buffer. Following primary antibody application, slides were washed and incubated in secondary antibodies (Table 3A) diluted in the blocking buffer for 1 h at room temperature. Slides were washed as above and stained with DAPI in PBS for 10 min at room temperature. Slides were washed as above and mounted with ProLong Gold Antifade Reagent. Stained cells were imaged with a ZEISS confocal microscope. Cell counts were reported as the average number of cells across 10 randomly selected hpf per well in each group.

### Senescence-Associated β-Galactosidase (SA-β-Gal) Staining

Cells were stained with β-galactosidase at pH 6.0 with Senescence β-galactosidase Staining Kit (Cell Signaling Technology, 9860), according to the manufacturer's protocol. Stained cells were imaged with a ZEISS confocal microscope.

### Flow Cytometry

Cells were washed once with PBS, including 5% newborn calf serum (Thermo Fisher Scientific, 26010074) and 0.01% sodium azide (Sigma-Aldrich, S2002-100G), and stained with antibodies (Tables 3B and 3B) on ice for 30 min followed by secondary antibody if needed (Table 3A). Following the surface marker staining, cells were fixed and permeabilized for intracellular staining using True-Nuclear Transcription Factor Buffer Set (BioLegend, 424401). Permeabilized cells were stained with antibodies (Table 3A) overnight at 4° C. Cells were washed and then examined by BD LSRFortessa X-20 flow cytometer (BD Bioscience, Billerica, MA). Data were analyzed using FlowJo software (BD Life Sciences, Franklin Lakes, NJ). Relative fluorescence intensity was calculated by subtracting the median fluorescence intensity of isotype control from the median fluorescence intensity of stained samples.

### Cytotoxicity Assay on Senescent Fibroblasts

Normal and senescent fibroblasts were seeded at a density of 5×10³ cells/well into 24 well plates, and were cultured in DMEM, including 10% FBS, 1% penicillin/streptomycin, 1% glutamine, overnight at 37° C. Fibroblasts were pre-treated with 100 μg/ml HLA-II blocking antibody (Bio X Cell, Lebanon, NH, BE0306) or 100 μg/ml isotype IgG antibody in the absence of FBS overnight at 37° C. Skin immune cells were subsequently added to each fibroblasts-placed well and co-cultured in the presence of 20 ng/ml human IL-2 recombinant and 20 ng/ml human IL-15 recombinant (BioLegend, 570304) in 24 well plates (Ratio of 50:1 immune cell-to-fibroblast). Following 6 h of co-culture, remaining adherent fibroblasts were fixed and stained as described in immunocytochemistry.

Human Skin T Cell Stimulation with Recombinant HCMV Proteins

Isolated human skin immune cells were treated with 10 μg/ml of recombinant HCMV-gB (Abcam, Cambridge, UK, ab43040) or 10 μg/ml of recombinant HCMV-gH (MyBioSource, San Diego, CA, MBS1138239) in the presence or absence of 1.67 μg/ml of Poly (I.C) (Thermo Fisher Scientific, tlrl-pic). After 20 h of incubation, Brefeldin A was added at the concentration of 5 μg/ml and cells were incubated for 4 h and collected for flow cytometric analysis. As a positive control, cells were treated with 81 nM phorbol 12-myristate 13-acetate (PMA) plus 1.34 UM Ionomycin (BioLegend, 423301) for 30 min. Cells were stained as described in flow cytometry.

### RNA In Situ Hybridization

RNA in situ hybridization was performed as previously described³⁷. Briefly, RNA in situ hybridization was performed on PFA-fixed paraffin-embedded tissue sections using the RNAscope 2.5 HD detection reagent protocol (Advanced Cell Diagnostics, Newark, CA) with accommodation to simultaneously stain for vimentin protein. 5 μm sections were baked at 60° C. for 60 min. Slides were treated with xylene, followed by 100% ethanol, and allowed to dry. Slides were treated with hydrogen peroxide at room temperature for 10 min and then washed with deionized water. Antigen retrieval was performed with RNAscope Target Retrieval Reagent (Advanced Cell Diagnostics, 322000) using a pressure cooker for 15 min. Slides were incubated with DNase-I (Sigma-Aldrich, D5319-500UG) at 37° C. for 30 min in a HybEZ Oven II (Advanced Cell Diagnostics, 321720) and then washed. RNAscope Protease Plus (Advanced Cell Diagnostics, 322331) treatment was applied at 40° C. for 15 min. After target probe amplification and hybridization steps, sections were stained with Fast RED reagent (RNAscope 2.5 HD Detection Reagents-RED, Advanced Cell Diagnostics, 322360). For hematoxylin staining, slides were washed with deionized water and then stained with hematoxylin (Sigma-Aldrich, GHS132-1L) for 1 min, followed by staining with 0.02% ammonium hydroxide (Ricca Chemical Company, Arlington, TX, 642-16). For immunofluorescent staining, slides were washed with deionized water and then PBS including 0.1% Tween 20. Slides were blocked with 5% goat serum and 5% bovine serum albumin in PBS, including 0.1% Tween 20, for 1 h at room temperature. Slides were stained as described in histology.

### Quantification of HCMV DNA

Quantification of HCMV DNA in human skin samples and fibroblasts was performed by quantitative real-time PCR as described previously^{38,39}. Following the kit instructions, DNA was isolated from fibroblasts using Quick-DNA/RNA Microprep Plus kit (Zymo Research, Irvine, CA, D7005). For human skin, DNA was isolated using a Direct-zol DNA/RNA Miniprep Kit (Zymo Research, R2080) following the kit instructions. Quantitative real-time PCR used the SYBRGreen format and HCMV primers detected the lower matrix phosphoprotein (UL83) gene. GAPDH was used as the internal control gene. Primer sets are described in Table 3F. PCR was performed on the 7500 Real-Time PCR System (Applied Biosystems, Inc., Foster City, CA) in a total volume of 25 μL in the presence of 5 μL of DNA sample, 12.5 μL of SYBRGreen PCR MasterMix (Bio-Rad, Hercules, CA, 1725121) and 250 nM of each of the primers. The temperature profile was 95° C. for 10 min, 40 cycles at 95° C. for 15 s and 60° C. for 60 s. At the end of each run, a melting curve analysis was performed. The melting temperature range for HCMV DNA positive samples was 81.5±0.5° C. Relative expression of UL83 was calculated by the 44Ct method.

### UVA Radiation

Fibroblasts were radiated through PBS with UVA (5 or 10 J) generated by a UVP XX-Series Bench Lamp, 115V (Thermo Fisher Scientific, UVP95004208), and cultured for one day or five days. Sham radiation was used as a negative control. Radiation intensity was measured using a light meter (InternationalLight Technologies, Peabody, MA, ILT2400).

### Human Cytomegalovirus (HCMV) Infection

Human cytomegalovirus, AD-169, was purchased from American Type Culture Collection (Manassas, VA, VR-538). Viral concentration was determined by plaque assay. Human dermal fetal fibroblasts were seeded at a density of 2-3×10⁴ cells/cm² and cultured as described in the culture of human dermal fibroblasts. One day after seeding, culture media was replaced with DMEM with 0.1% bovine serum albumin, and the cells were infected with a multiplicity of infection (MOI) of 1. Five days after infection, DNA was isolated from the cells. Three days after infection, infected cells were stained for immunocytochemistry as described above.

### RNA-Seq Analysis

Human skin tissues were homogenized with RLT buffer (Qiagen, Hilden, Germany, 79216) supplemented with 1% β-mercaptoethanol (Thermo Fisher Scientific, 21-985-023). Full-length cDNA and sequencing libraries were prepared from 1 ng RNA using the Smart-Seq2 protocol as previously described³³. Libraries were sequenced on a Novaseq 6000 (Illumina) through the Broad Genomics Platform. The FASTQ files were aligned to the human genome/hg19 (GENCODE v19) by STAR-2.5.1b³⁴. Aligned transcripts were quantified by using RSEM-1.2.3.1³⁵. Differentially expressed genes (DEG) were analyzed by DESEq2³⁶. Cultured fibroblasts were prepared in TCL buffer (Qiagen, 1031576) supplemented with 1% β-mercaptoethanol. Then, each sample was added into a 96-well Eppendorf twin-tec barcoded plate provided by the Broad Institute (Cambridge, MA). Modified SmartSeq2 complementary DNA and Illumina Nextera XT library construction and sequencing were conducted at the Broad Institute using the Illumina NextSeq 500 System. The quality of FASTQ files was examined using FastQC-0.11.8. The sequences were mapped to the human genome/GRCh38 using STAR-2.5.334 Sequences located at transcripts were quantified by using RSEM-1.3.135. DEGs were analyzed using DESEq2-1.24.036. Original data are available in the NCBI Gene Expression Omnibus (GEO) with accession number GSE191055.

10× Genomics Sample Processing and cDNA Library Preparation

The 10× Genomics Chromium Next GEM Single Cell 30 Reagents Kits v3.1 (Dual Index) user guide (support. 10×genomics.com/single-cell-gene-expression/library-prep/doc/user-guide-chromium-single-cell-3-reagent-kits-user-guide-v31-chemistry-dualindex) was used to prepare the single cell suspension. Cells were isolated from young and old normal breast skin using the Whole Skin Dissociation Kit (Miltenyi Biotec, Bergisch Gladbach, Germany, Cat 130-101-540). The kit's instructions were followed, and enzyme P was excluded to see CD4 and CD8 epitopes. After isolating cells, the single-cell samples were passed through a 40 mm cell strainer. The cells were stained and sorted CD45⁻PI⁻ cells using SH800 Cell Sorter (Sony Biotechnology Inc., San Jose, CA). After sorting, the number of cells was counted using a Cellometer Auto 2000, Slide SD100 (Nexcelom) and VitaStain AOPI Staining Solution (Nexcelom, CS2-010-5 ml). Then, the appropriate volume of each sample was diluted to recover 6,000-10,000 skin-isolated CD45⁺ cells. Subsequently, the single-cell suspension, Gel Beads, and oils were added to the 10× Genomics single-cell G chip. After droplet generation, samples were transferred into PCR tubes, and reverse transcription was performed using a C1000 Touch Thermal Cycler (Bio-Rad). After reverse transcription, cDNA was recovered using a recovery agent provided by 10× Genomics, followed by silane DynaBead clean-up as outlined in the user guide. Before clean-up using SPRIselect beads, the cDNA was amplified for 11-12 cycles depending on the number of the targeted cell recovery. The cDNA concentration was detected by a Qubit 4 Fluorometer (Invitrogen, Q32856) and Qubit 1× dsDNA HS Assay Kit (Invitrogen, Q33230). The mean peak size was obtained using Agilent 4200 TapeStation (Agilent Technologies, Santa Clara, CA, G2991BA) and Agilent High Sensitivity D5000 ScreenTape Assay (Agilent Technologies, 5067-5592, 5593). The skin-isolated cells cDNA libraries were prepared referring to the Chromium Next GEM Single Cell 30 Reagent Kits v3.1 user guide. After libraries were prepared, cDNA concentration was rechecked with Qubit 1× dsDNA HS Assay Kit and KAPA Library Quantification Kit (Roche, Basel, Switzerland, KK4835), and the mean peak size was obtained using Agilent High Sensitivity D1000 ScreenTape Assay (Agilent Technologies, 5067-5584, 5585).

### ScRNA-seq Analysis

Libraries were sequenced using NextSeq 500/550 Hi Output Kit v2.5 150 cycle (illumina, 20024907). Libraries were sequenced using NextSeq 500/550 High Output Kit v2.5 150 cycle (illumina, 20024907) with NextSeq 550 (Illumina). The raw binary base call (BCL) sequence was converted to FASTQ files using Cell Ranger-6.0 (10× Genomics) mkfastq with default parameters. The FASTQ files were mapped using human reference (GRCh38) with Cell ranger-6.0 count. The matrix data were analyzed using Seurat-4.3.0 in R-4.2.2.60 Low-quality cells with fewer than 20 genes, more than 3000 genes, or more than 15% of mitochondrial genes were removed from further analysis. “NormalizeData” followed by the “ScaleData” function was used to normalize and scale the sequencing reads. To remove the batch effects among samples, the top 2000 highly variable genes were identified from each sample, and then the repeatedly variable genes across samples were integrated using “SelectIntegrationFeatures” for principal component analysis (PCA) analysis. “FindIntegrationAnchors” was used to identify anchors, and then they were integrated into the data using the “IntegrateData” function. “ScaleData,” “RunPCA,” “RunUMAP,” “FindNeighbors,” and “Findclusters” were used to obtain Uniform Manifold Approximation and Projection (UMAP) plot. CD4′ T cell clusters were defined with the criteria of CD3D⁺TRAC⁺TRDC⁻ CD8A⁻. “FindMarkers” with DEseq2 parameter was used to calculate differentially expressed genes (DEGs) between conventional CD4 (PRF1⁻, CD8A⁻) and CD4 CTL (PRF1⁺ CD8A⁻NCAM⁻). CD8 CTL was defined using PRF1⁺ CD8A⁺. Original data are available in the NCBI Gene Expression Omnibus (GEO) with accession number GSE221232.

siRNA Transfection

Normal fibroblasts and senescent fibroblasts were seeded at a density of 4×10⁴ cells into a 10 cm dish. One day after seeding, the cells were collected and transfected Human CIITA siRNA SMARTPool (Horizon Discovery, Waterbeach, UK, L-011083-00-0005) to senescent fibroblasts using an NHDF Nucleofector kit (Lonza, VPD-1001) and Nucleofector 2b Device (Lonza, AAB-1001). Negative Control siRNA (Qiagen, 1022076) was used for negative control. After transfection, fibroblasts were reseeded at a density of 2.5×10³ cells/well into chamber slide glasses (CELLTREAT Scientific Products). 48 h after transfection, cells were collected for flow cytometric analysis. For the cytotoxic assay, fibroblasts were co-cultured with skin-isolated cells (E:T ratio=50:1) and incubated for 6 h. After co-culture, fibroblasts were fixed and stained as described in immunocytochemistry.

Characterization of Skin-Isolated Immune Cells Co-Cultured with Senescent Fibroblasts

Normal fibroblasts and senescent fibroblasts were seeded at a density of 2×10³ cells/well into 48-well plates. One day after seeding, skin-isolated cells were added at a density of 3×10⁴ cells/well. After 20 h of incubation, brefeldin A was added at the concentration of 5 μg/ml, and cells were incubated for 4 h and collected for flow cytometric analysis. Cells were stained as described in flow cytometry to detect CD137 and IFNγ expression. To detect CD107a expression, skin-isolated cells were used. After 1 h of co-culture, brefeldin A (BioLegend, 420601, 5 μg/ml) and monensin (BioLegend, 420701, 2 μM) was added with CD107a-FITC antibody (Dilution: 200). 4 h later, cells were collected for flow cytometric analysis.

### Trans-Well Migration Assay

Skin-isolated cells were seeded at a density of 1.5×10⁴ cells/well into the top inserts of the 24-well trans-well plate (Corning, 07-200-149). The pore size is 5 μm. RPMI containing recombinant human CXCL9 (PeproTech, 300-26) or PBS (carrier only) at the concentration of 50 nM was added into the bottom well. Skin-isolated cells were incubated for 60 min. Then, cells were counted and stained as described in the flow cytometry section.

### HCMV Epitope-Specific CD4 CTL Detection

The HCMV-gB-specific DRB1*0701 DYSNTHSTRYV phycoerythrin (PE)-conjugated tetramer was generated by the National Institutes of Health (NIH) tetramer core facility at Emory University, based on previous publications.⁶¹-65 The NIH also provided a DRB1*0701 PE-conjugated control peptide (PVSKMRMATPLLMQA) tetramer. DYSNTHSTRYV is an HCMV-gB epitope sequence, and it was previously shown to be recognized by human CD4 CTL.⁶⁶ Peptide DYSNTHSTRYV is provided by GenScript (Piscataway, NJ). Cells were incubated with test or control tetramer at the concentration of 5.6 μg/ml at 37° C. in the dark for 3 h. After repeated washing, cells were stained as described in the flow cytometry section.

### HLA-DRB1*07 Typing

DNA isolation from flash-frozen tissue was performed using the Qiagen DNeasy Blood and Tissue Kit (Qiagen, 69504) according to the manufacturer's instructions. A NanoDrop spectrophotometer (NanoDrop Technologies, Wilmington, DE, ND-1000) was used to determine DNA quality and concentration. HLA-DRB1*07 typing experiments were performed according to the manufacturer's instructions of Olerup SSP (CareDx, Brisbane, CA, 101.118-24u). Briefly, DNA, mastermix, and Platinum TaqDNA Polymerase (Invitrogen, 10966-026) were added to appropriate primer cocktails. The reaction mix was placed on a thermocycler; samples were run on 1% agarose (Denville Scientific, Metuchen, NJ, GR140-500) gels and then imaged. The kit typed for DRB1*07:01 to DRB1*07:123, recognized by the HLA Nomenclature Committee in April 2021, using a total of 21 reactions and 1 control reaction.

### TCR Sequencing and Analysis

DNA isolation, concentration, and purity determination were performed as above. Adaptive Biotechnologies (Seattle, WA) performed high-throughput sequencing of the provided DNA and produced the raw data.

### SenTraGor Staining

SenTraGor staining was done on a formalin-fixed paraffin-embedded (FFPE) slide following the manufacturer's instruction. Briefly, after deparaffinization and rehydration, SenTraGor reagent incubation was done at room temperature for 3 min. After washing, slides were incubated with mouse anti-biotin antibody (Abcam, ab201341) with p16^{INK4a} and vimentin antibody overnight at 4° C. Secondary antibody staining was done as described above.

### SCFA-Mediated CD4⁺ T Cell Stimulation Assay

T cells were seeded at a density of 2.5×10⁵ cells into 60 mm dish, and were added to each 5 mM SCFAs, including butylate (EMD Millipore), pentanoate (Ambeed Inc), or propionate (Sigma-Aldrich) with 20 ng/ml human IL-2 recombinant and 20 ng/ml human IL-15 recombinant for 2 days. Following 2 days of co-incubation, T cells were subsequently added to each senescent fibroblasts-placed wells and were cultured in T cell medium with 20 ng/ml human IL-2 recombinant and 20 ng/ml human IL-15 recombinant in 24 well plate (Ratios of 50:1 T cells-to fibroblasts respectively). Following 6 hr of co-incubation, remaining fibroblasts were stained with vimentin and cleaved caspase-3 to examine the frequency of T cell-mediated cytotoxicity against senescent fibroblasts.

### Statistical Analysis

All bar graphs and dot plots show mean±SD. The two-tailed Mann-Whitney U test was used as the significance test for cell counts in human skin histological analysis. A two-tailed paired t-test was used for the cultured dermal fibroblasts assays. The Student's t-test for the Pearson correlation coefficient was used as the significance test for the linear regression in the scatter plots. One-way ANOVA with Tukey's multiple comparison test was used for the co-culture cytotoxic assay. One-way ANOVA with Dunnett's multiple comparison test was used for the HCMV-gB 10 stimulation assay. The chi-square test was used as the test of significance for categorical variables. Prism 9 was used for statistical analysis. P value <0.05 was considered significant.

TABLE 3A
Antibodies

| | | |
|---|---|---|
| REAGENT or | | |
| RESOURCE | SOURCE | IDENTIFIER |

Antibodies

| | | |
|---|---|---|
| CD3 | Abcam | Cat#ab11089; |
| | | RRID:AB_2889189 |
| CD4 | ThermoFisher | Cat#MA1-39582; |
| | Scientific | RRID:AB_10986805 |
| Perforin | R&D Systems | Cat#MAB8011 |
| T-bet | Abcam | Cat#ab91109; |
| | | RRID:AB_2050371 |
| GATA3 | BioCare Medical | Cat#CM405A |
| ROR-gt | BioCare Medical | Cat#ACI 3208 A |
| Foxp3 | eBioscience | Cat#14-4776; |
| | | RRID:AB_467554 |
| CD8 | Cell Signaling | Cat#70306 |
| | Technology | |
| TCR gd | Thermo Fisher | Cat#TCR1061; |
| | Scientific | RRID:AB_223500 |
| CD56 | Cell Signaling | Cat#3576; |
| | Technology | RRID:AB_2149540 |
| CD11c | Abcam | Cat#ab52632; |
| | | RRID:AB_2129793 |
| CD141 | Bio-Rad | Cat#MCA641T; |
| | | RRID:AB_2287220 |
| CD1c | Abcam | Cat#ab156708; |
| | | RRID:AB_2889187 |
| CD207 | Novus Biologicals | Cat#DDX0362P; |
| | | RRID:AB_2892751 |
| CD1a | Dako | Cat#M3571; |
| | | RRID:AB_2073290 |
| CD68 | Abcam | Cat#ab955; |
| | | RRID:AB_307338 |
| CD86 | Cell Signaling | Cat#91882; |
| | Technology | RRID:AB_2797422 |
| CD206 | Abcam | Cat#ab64693; |
| | | RRID:AB_1523910 |
| Mast Cell Tryptase | Dako | Cat#M705229-2 |
| Neutrophil Elastase | Abcam | Cat#ab68672; |
| | | RRID:AB_1658868 |
| CXCL9 | Abcam | Cat#ab9720; |
| | | RRID:AB_308764 |
| HCMV-gB | Abcam | Cat#ab54023; |
| | | RRID:AB_879780 |
| IILA-DP, DQ, DR | Dako | Cat#M0775; |
| | | RRID:AB_2313661 |
| ₚ₁₆INK4a | Abcam | Cat#ab108349; |
| | | RRID:AB_10858268 |
| Vimentin | Abcam | Cat#ab8978; |
| | | RRID:AB_306907 |
| pan-HCMV | Sigma-Aldrich | Cat#213M-24-ASR |
| HCMV IE1/2 | Abcam | Cat#ab53495; |
| | | RRID:AB_882995 |
| PDGFRa | Cell Signaling | Cat#3174; |
| | Technology | RRID:AB_2162345 |
| p21 Waf1/Cip1 | Cell Signaling | Cat#2947; |
| | Technology | RRID:AB_823586 |
| ULBP2 | Cell Signaling | N/A |
| | Technology | |
| Vimentin | Abcam | Cat#ab24525; |
| | | RRID:AB_778824 |
| Cleaved caspase-3 | Cell Signaling | Cat#9664; |
| | Technology | RRID:AB_2070042 |
| Rab5 | Abcam | Cat#ab13253; |
| | | RRID:AB_299796 |
| Goat anti | Thermo Fisher | Cat#A-21449; |
| Chicken IgY (H + L) | Scientific | RRID:AB_1500594 |
| Secondary | | |
| antibody Alexa | | |
| Fluor 647 | | |
| Goat anti | Thermo Fisher | Cat#A-11001; |
| Mouse IgG (H + L) | Scientific | RRID:AB_2534069 |
| Cross Adsorbed | | |
| Secondary | | |
| Antibody, Alexa | | |
| Fluor 488 | | |
| Goat anti | Thermo Fisher | Cat#A-11036; |
| Rabbit IgG | Scientific | RRID:AB_10563566 |
| (H + L) Cross | | |
| Adsorbed | | |
| Secondary | | |
| Antibody, | | |
| Alexa Fluor 568 | | |
| Goat anti Rat | Thermo Fisher | Cat#A-21247; |
| IgG (H + L) Secondary | Scientific | RRID:AB_141778 |
| Antibody, Alexa | | |
| Fluor 647 | | |
| Goat anti Rabbit | Thermo Fisher | Cat#A-11008; |
| IgG (H + L) Cross | Scientific | RRID:AB_143165 |
| Adsorbed | | |
| Secondary | | |
| Antibody, | | |
| Alexa Fluor 488 | | |
| Goat anti | Thermo Fisher | Cat#A-11034; |
| Rabbit IgG (H + L) | Scientific | RRID:AB_2576217 |
| Secondary | | |
| Antibody, | | |
| Alexa Fluor | | |
| 488 | | |
| HLA-DR, DP, DQ | BioLegend | Cat#361715 |
| | | Clone: Tü 39; |
| | | RRID:AB_2750317 |
| CD3 | BioLegend | Cat#300405 |
| | | Clone: UCHT1; |
| | | RRID:AB_314059 |
| CD4 | Thermo Fisher | Cat#45-0048-41 |
| | Scientific | Clone: OKT4; |
| | | RRID:AB_10718242 |
| CD8 | BioLegend | Cat#344731 |
| | | Clone: SK1; |
| | | RRID:AB_2564623 |
| CD45 | BioLegend | Cat#304041 |
| | | Clone: HI30; |
| | | RRID:AB_2562105 |
| CD45 | BioLegend | Cat#304011 |
| | | Clone: HI30; |
| | | RRID:AB_314399 |
| CD69 | BioLegend | Cat#310929 |
| | | Clone: FN50; |
| | | RRID:AB_10933255 |
| Perforin | BioLegend | Cat#308127 |
| | | Clone: dG9; |
| | | RRID:AB_2572050 |
| Perforin | BioLegend | Cat#308126 |
| | | Clone: dG9; |
| | | RRID:AB_2572049 |
| IFNg | Thermo Fisher | Cat#17-7319-82 |
| | Scientific | Clone: 4S.B3; |
| | | RRID:AB_469506 |
| CD107a | BioLegend | Cat#328605 |
| | | Clone: H4A3; |
| | | RRID:AB_1186058 |
| CD137 | BD Biosciences | Cat#745737 |
| | | Clone: 4B4-1; |
| | | RRID:AB_2743209 |
| CXCR3 (CD183) | BioLegend | Cat#353706 |
| | | Clone: G025H7; |
| | | RRID:AB_10962912 |
| HLA-II blocking | Bio X Cell | Cat#BE0306; |
| antibody | | RRID:AB_2736986 |
| HCMV-gB-specific | NIH Tetramer core | N/A |
| DRB1 PE- | | |
| conjugated | | |
| tetramer | | |
| DRB1 control | NIH Tetramer core | N/A |
| peptide PE- | | |
| conjugated | | |
| tetramer | | |
| Anti-biotin | Abcam | Cat#ab201341 |
| | | Clone: Hyb-8; |
| antibody | | RRID:AB_2861249 |

TABLE 3B
Bacterial and virus strains

| | | |
|---|---|---|
| REAGENT or RESOURCE | SOURCE | IDENTIFIER |

Bacterial and virus strains

| | | |
|---|---|---|
| Human cytomegalovirus | ATCC | VR-538 |
| AD-169 | | |
| Biological samples | | |
| De-identified skin | Massachusetts General | Protocol: |
| samples | Hospital | 2015P002068 |

TABLE 3C
Chemicals, peptides, and recombinant proteins

| | | |
|---|---|---|
| REAGENT or RESOURCE | SOURCE | IDENTIFIER |

Chemicals, peptides, and recombinant proteins

| | | |
|---|---|---|
| Zombie NIR Fixable | BioLegend | Cat#423106 |
| Viability Kit | | |
| Propidium iodide | Sigma-Aldrich | Cat#P4864-10ml |
| Recombinant | PeproTech | Cat#300-26 |
| human CXCL9 | | |
| dispase | Stemcell | Cat#07913 |
| Collagenase/hyaluronidase | Stemcell | Cat#07912 |
| DMEM | Thermo Fisher | Cat#11-965-118 |
| | Scientific | |
| RPMI 1640 | Thermo Fisher | Cat#21-870-092 |
| | Scientific | |
| DNase-I | Sigma-Aldrich | Cat#101041 |
| Collagenase-I | Thermo Fisher | Cat#LS004196 |
| | Scientific | |
| 2-Mercaptoethanol | Sigma-Aldrich | Cat#M6260-100ML |
| Recombinant human IL-2 | BioLegend | Cat#589104 |
| Triton-X | Thermo Fisher | Cat#BP151 |
| | Scientific | |
| Citrate-Based Antigen | Vector | Cat#H-3300-250 |
| Unmasking Solution | Laboratories | |
| Tween 20 | Sigma-Aldrich | Cat#P1379 |
| Normal goat serum | Sigma-Aldrich | Cat#G9023 |
| Bovine serum albumin | Thermo Fisher | Cat#BP1600 |
| | Scientific | |
| DAPI | Invitrogen | Cat#D3571 |
| ProLong Gold | Thermo Fisher | Cat#P36930 |
| Antifade Reagent | Scientific | |
| RLT buffer | Qiagen | Cat#79216 |
| TCL buffer | Qiagen | Cat#1031576 |
| Newborn calf serum | Thermo Fisher | Cat#26010074 |
| | Scientific | |
| Sodium azide | Sigma-Aldrich | Cat#S2002-100G |
| True-Nuclear | BioLegend | Cat#424401 |
| Transcription Factor | | |
| Buffer Set | | |
| Recombinant human IL-15 | BioLegend | Cat#570304 |
| Recombinant human | Abcam | Cat#ab43040 |
| cytomegalovirus | | |
| glycoprotein B | | |
| Recombinant human | MyBio Source | Cat#MBS1138239 |
| cytomegalovirus | | |
| glycoprotein H | | |
| Cell Activation Cocktail | BioLegend | Cat#423301 |
| without Brefeldin A | | |
| (PMA and ionomycin) | | |
| Brefeldin A | BioLegend | Cat#420601 |
| monensin | BioLegend | Cat#420701 |
| RNAscope Target | Advanced Cell | Cat#322000 |
| Retrieval Reagent | Diagnostic | |
| DNase-I | Sigma-Aldrich | Cat#D5319-500UG |
| HybEZ Oven II | Advanced Cell | Cat#321720 |
| | Diagnostic | |
| RNAscope Protease | Advanced Cell | Cat#322331 |
| Plus | Diagnostic | |
| Fast RED reagent | Advanced Cell | Cat#322360 |
| | Diagnostic | |
| Hematoxylin | Sigma-Aldrich | Cat#GHS132-1L |
| Ammonium | Ricca Chemical | Cat#642-16 |
| hydroxide | Company | |
| SYBRGreen PCR | Bio-Rad | Cat#1725121 |
| MasterMix | | |
| Slide SD100 | Nexcelom | Cat#CHT4-SD100-002 |
| VitaStain AOPI | Nexcelom | Cat#CS2-010-5ml |
| Staining Solution | | |
| SPRIselect | Beckman Coulter | Cat#B23317 |
| Qubit 1X dsDNA | Invitrogen | Cat#Q33230 |
| HS Assay Kit | | |
| Agilent High | Agilent | Cat#5067-5592, 5593 |
| Sensitivity D5000 | | |
| Screen Tape Assay | Technologies | |
| Agilent High | Agilent | 5067-5584, 5585 |
| Sensitivity D1000 | | |
| ScreenTape Assay | Technologies | |
| Peptide DYSNTHSTRYV | Genscript | N/A |
| Platinum TaqDNA | Invitrogen | Cat#10966-026 |
| Polymerase | | |
| agarose | Denville Scientific | Cat#GR140-500 |
| SenTraGor Cell | Cayman Chemical | Cat#35568 |
| Senescence Reagent | Company | |

TABLE 3D
Commercial Assays

| | | |
|---|---|---|
| REAGENT or RESOURCE | SOURCE | IDENTIFIER |

Commercial assays

| | | |
|---|---|---|
| Chroium Next GEM Single Cell 3′ | 10x Genomics | PN-1000269 |
| Kit v3.1 | | |
| Chromium Next GEM Chip G | 10x Genomics | PN-10000127 |
| Single Cell Kit | | |
| Dual Index Kit TT Set A | 10x Genomics | PN-10000215 |
| NextSeq 500/550 Hi Output Kit | illumina | 20024907 |
| v2.5 150 cycle | | |
| Senescence b-galactosidase Staining | Cell Signaling | Cat#9860 |
| Kit | Technology | |
| Quick-DNA/RNA Microprep Plus | Zymo Research | Cat#D7005 |
| Kit | | |
| Direct-zol DNA/RNA miniprep Kit | Zymo Research | Cat#R2080 |
| W | | |
| hole Skin Dissociation Kit | Miltenyi Biotec | Cat#130-101-540 |
| NHDF Nucleofector kit | Lonza | Cat#VPD-1001 |
| DNeasy Blood and Tissue Kit | Qiagen | Cat#69504 |
| Olerup SSP HLA typing kit | CareDx | Cat#101.118-24u |

TABLE 3E
Deposited Data and Experimental Models

| | | |
|---|---|---|
| REAGENT or RESOURCE | SOURCE | IDENTIFIER |

Deposited data

| | | |
|---|---|---|
| RNA Sequencing | This work | Available at NCBI, |
| datasets | | GEO: GSE191055 |
| Single cell RNA | This work | Available at NCBI, |
| Sequencing datasets | | GEO: GSE221232 |

Experimental models: Cell lines

| | | |
|---|---|---|
| Neonatal dermal | Lonza | CC-2509 |
| fibroblasts | | |
| Human fetal dermal | ScienCellResearch | 2300 |
| fibroblasts | Laboratories | |

TABLE 3F
Oligonucleotides

| | | |
|---|---|---|
| REAGENT or RESOURCE | SOURCE | IDENTIFIER |

Oligonucleotides

| | | |
|---|---|---|
| GAPDH primer forward: | IDT | N/A |
| GGCTCTTAAAAAGTGCAGGGT | | |
| C (SEQ ID NO: 1) | | |
| GAPDH primer reverse: | IDT | N/A |
| GCTGTAGCCAAATTCGTTGTC | | |
| A (SEQ ID NO: 2) | | |
| UL83 primer forward: | IDT | N/A |
| TCTCGCACATCATGCTGGAT | | |
| (SEQ ID NO: 3) | | |
| UL83 primer reverse: | IDT | N/A |
| CGTTCATCAACAGGTTACCT | | |
| GAGAT (SEQ ID NO: 4) | | |
| Human CIITA siRNA | Horizon | Cat#L-011083- |
| SMARTPool | Discovery | 00-0005 |
| Negative Control | Qiagen | Cat#1022076 |
| siRNA | | |

TABLE 3G
Software and Algorithms

| | | |
|---|---|---|
| REAGENT or | | |
| RESOURCE | SOURCE | IDENTIFIER |

Software and algorithms

| | | |
|---|---|---|
| Zen Blue 3 | Zeiss | zeiss.com/microscopy/ |
| | | en/products/software/ |
| | | zeiss-zen.html |
| FlowJo 10 | BD Bioscience | flowjo.co |
| Prism 9 | GraphPad | graphpad.com/ |
| | | scientific-software/ |
| | | prism/ |
| Biorender | Biorender | biorender.com/ |
| DESeq2 | Bioconductor | bioconductor.org/ |
| | | packages/release/ |
| | | bioc/html/DESeq2.html |
| Seurat-4.3.0 | Satija Lab | satijalab.org/seurat/ |
| | | articles/pbmc3k_{—} |
| | | tutorial.html |
| Cell | 10x Genomics | support.10xgenomics.com/ |
| | | single-cell-gene-expression/ |
| Ranger-6.0 | | software/pipelines/ |
| | | latest/using/tutorial_ov |

Other

| | | |
|---|---|---|
| Zeiss Axio | Zeiss | N/A |
| Observer Z1 | | |
| Zeiss Axio | Zeiss | N/A |
| Scan.Z1 | | |
| Novaseq 6000 | Illumine | N/A |
| LSRFortessa | BD Bioscience | N/A |
| X-20 | | |
| 7500 Real- | Applied | N/A |
| Time PCR | Biosystems | |
| System | | |
| UVP XX-Series | ThermoFisher | UVP95004208 |
| Bench Lamp | Scientific | |
| 115V | | |
| Hand-Held | InternationalLight | ILT2400 |
| Light Meter | Technologies | |
| SH800 Cell | Sony | N/A |
| Sorter | Biotechnology | |
| Cellometer | Nexcelom | N/A |
| Auto 2000 | | |
| C1000 Touch | Bio-Rad | N/A |
| Thermal Cycler | | |
| Qubit 4 | Invitrogen | Q32856 |
| Fluorometer | | |
| Agilent 4200 | Agilent | G2991BA |
| TapeStation | Technologies | |
| Nucleofector | Lonza | AAB-1001 |
| 2b Device | | |
| NanoDrop | NanoDrop | ND-1000 |
| spectrophotometer | Technologies | |

### Example 1. Senescent Cells are Increased in the Old Human Skin

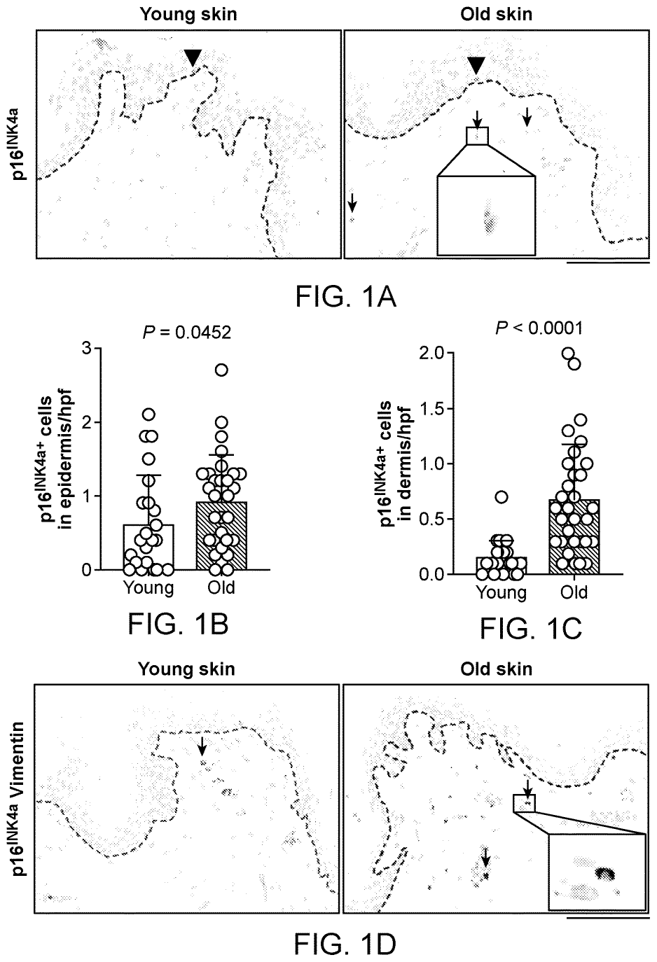

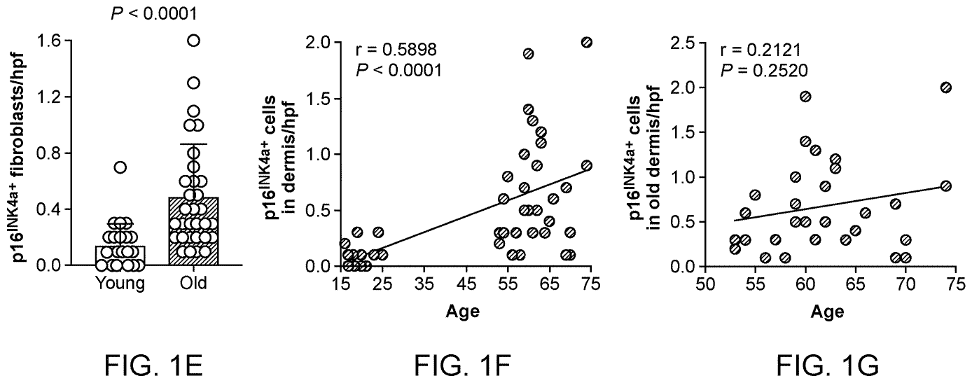

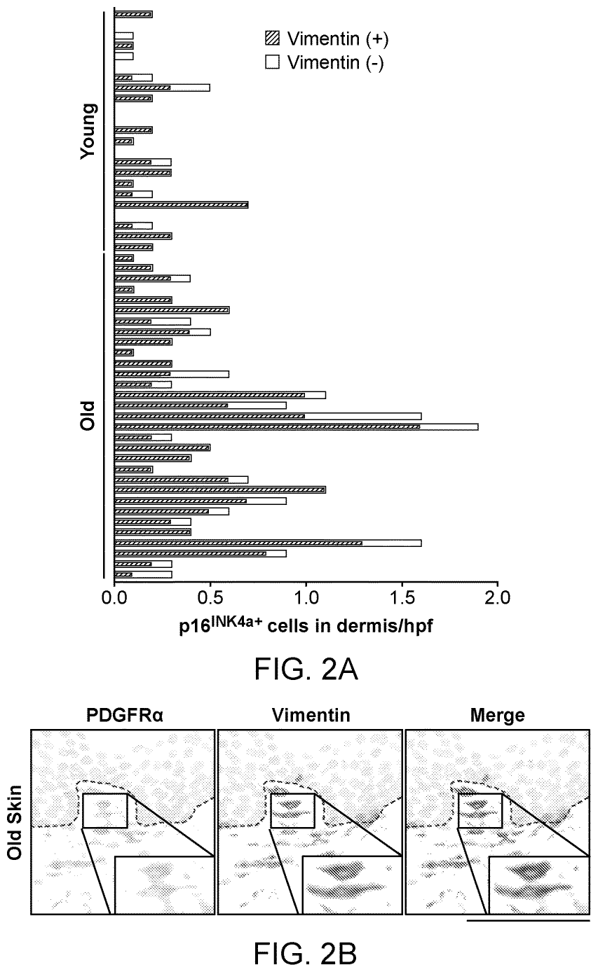

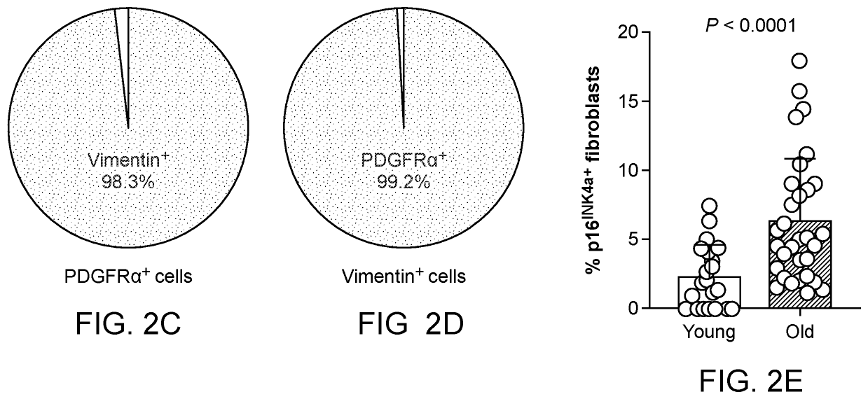

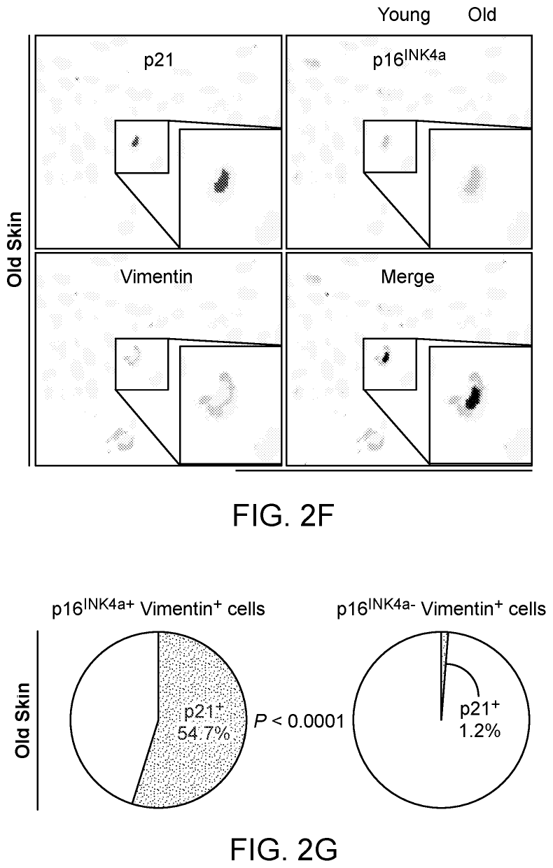

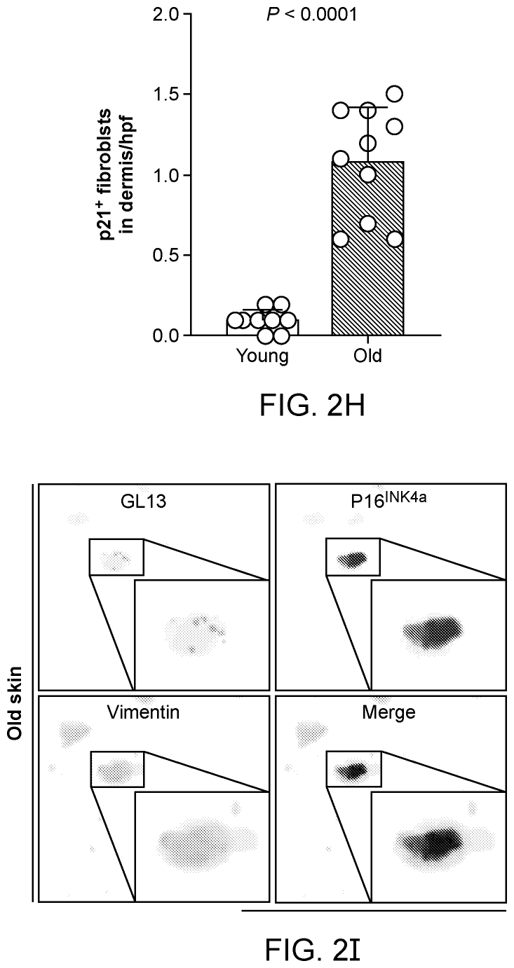

Over 800 human skin samples from various ages and anatomical sites were screed and identified cohorts of sun-protected truncal skin from young (n=23, average age: 23.1) and old (n=31, average age: 62.1) women (Table 1). Reduced epidermal thickness in the old versus young skin demonstrated the biological evidence of skin aging in the selected skin samples (Table 1). These skin cohorts enabled the evaluation of age-associated senescent cell accumulation in a human organ while excluding confounding factors like ultraviolet (UV) radiation, anatomic site variations, gender differences, and hair follicle density (Table 1). To determine whether the number of senescent cells changed with age, human skin samples were stained with p16^{INK4a}, a marker of cellular senescence^{1,18}. The number of p16^{INK4a} positive cells was significantly increased in the epidermis and dermis with age; however, the magnitude of this increase was significantly higher in the dermis (FIG. 1A-C). Over 80% of dermal p16^{INK4a} positive cells expressed vimentin, and most vimentin-positive dermal cells were also platelet-derived growth factor receptor a (PDGFRα) positive, indicating that most of the senescent cells in the old human dermis were fibroblasts (FIGS. 2A-2D). Accordingly, the number and percentage of p16^{INK4a+} vimentin⁺ fibroblasts were significantly increased in the old compared with young skin (FIGS. 1D, 1E, and 2E). The majority of p16^{INK4a+} dermal fibroblasts in the old skin expressed p21, which is upregulated during early cellular senescence, 67 while p21 expression was negligible in p16^{INK4a}-dermal fibroblasts (FIGS. 2F and 2G). Likewise, the numbers of p21⁺ vimentin⁺ fibroblasts was significantly increased in the old skin compared with young skin (FIG. 2H); p16^{INK4a+} dermal fibroblasts in the old skin were also positive SenTraGor (GL13), a marker for lipofuscin accumulation associated with cellular senescence (FIG. 2I).71 The number of dermal senescent cells was positively correlated (r=0.5898) with age across the young and old skin samples (P<0.0001; FIG. 1F). However, the number of dermal senescent cells did not show any significant correlation (r=0.2121) with advancing age within the old skin cohort (P=0.2520; FIG. 1G). This unexpected finding suggests that biological factor(s) other than increasing age may govern the accumulation of senescent cells in the elderly.

TABLE 1
Human skin samples information.

| | Young skin | | Old skin | | |
|---|---|---|---|---|---|
| | (n = 23) | | (n = 31) | | P value |
| Age, years | | | | | |
| Mean (SD) | 23.1 | (3.4) | 62.1 | (5.9) | <0.0001 |
| Range | 16-28 | | 53-74 | | |
| Gender, number (%) | | | | | — |
| Male | 0 | (0) | 0 | (0) | |
| Female | 23 | (100) | 31 | (100) | |
| Epidermal thickness, | 41.5 | (6.3) | 33.8 | (2.7) | 0.0006 |
| mean (SD), μm | | | | | |
| No. of hair follicle | 0.19 | (0.16) | 0.15 | (0.16) | 0.346 |
| units per mm skin, | | | | | |
| mean (SD) | | | | | |

### Example 2, CD4 CTL are Prominent Cytotoxic Lymphocytes in the Aging Human Skin

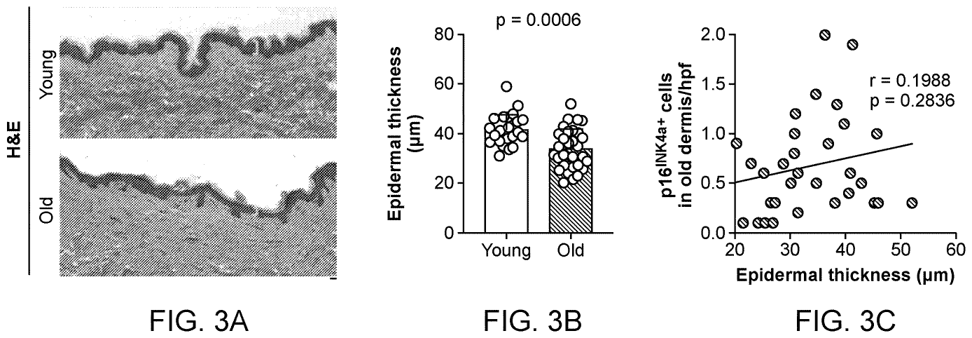

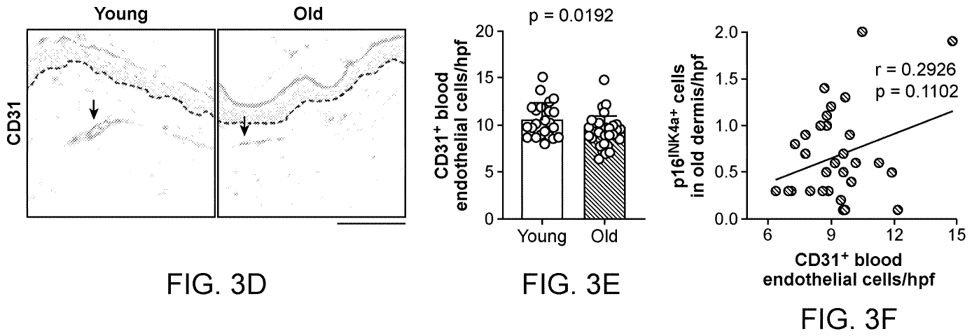

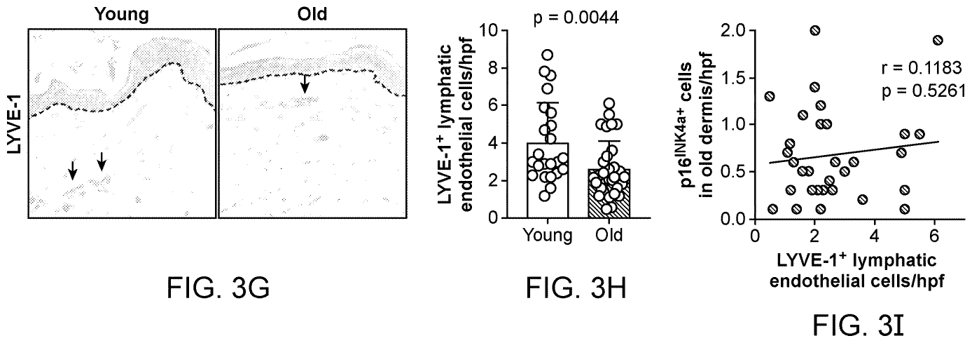

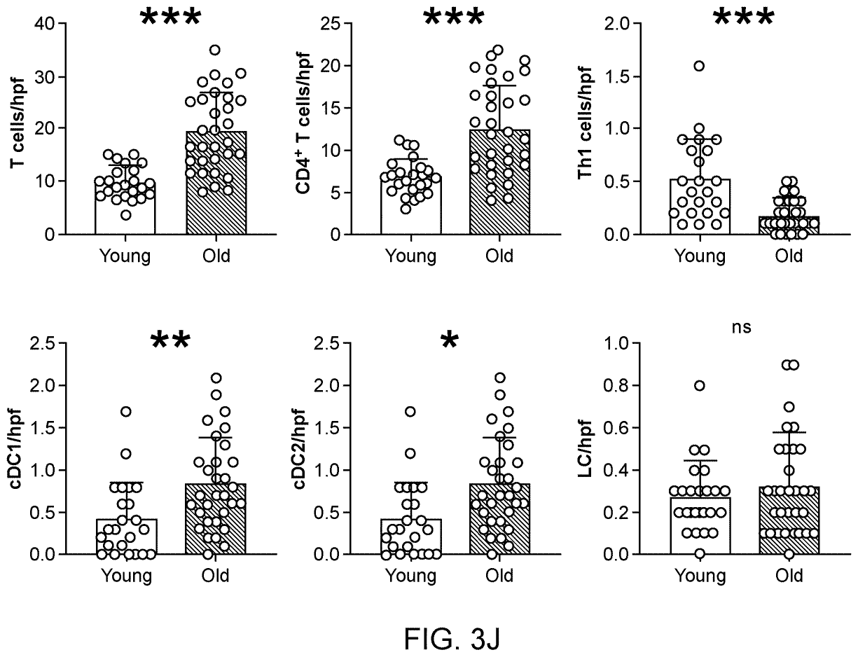

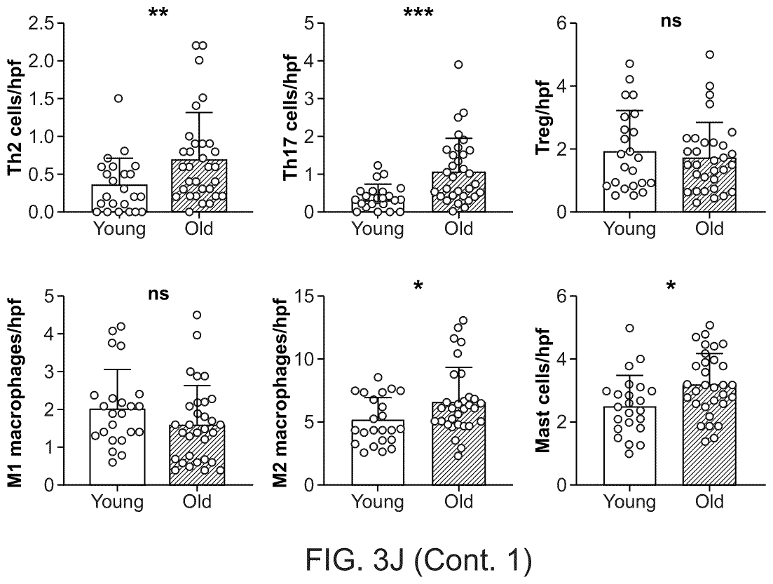

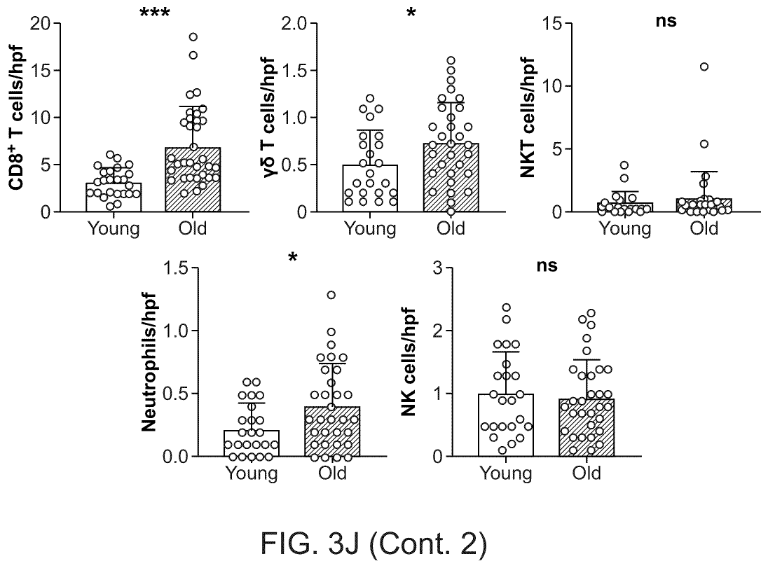

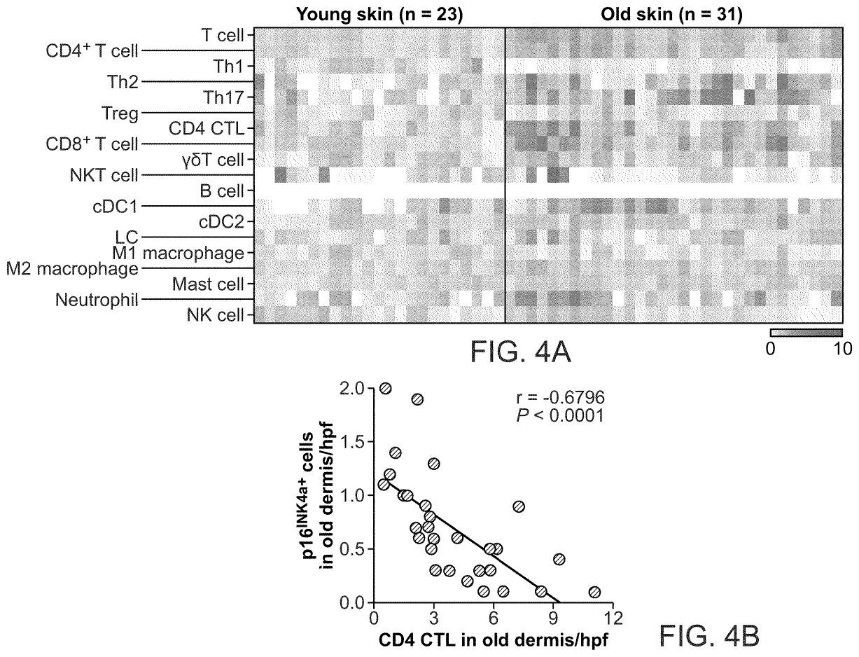

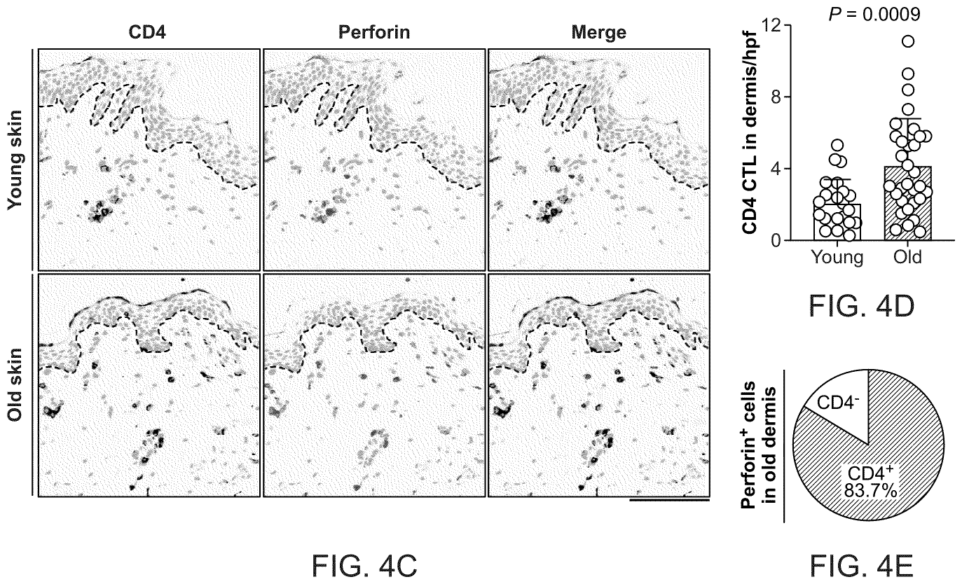

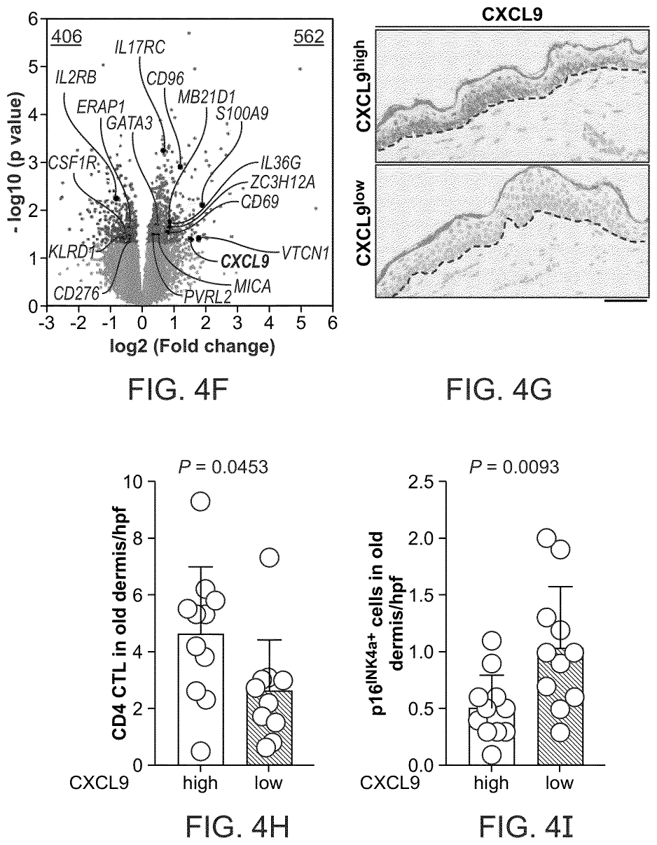

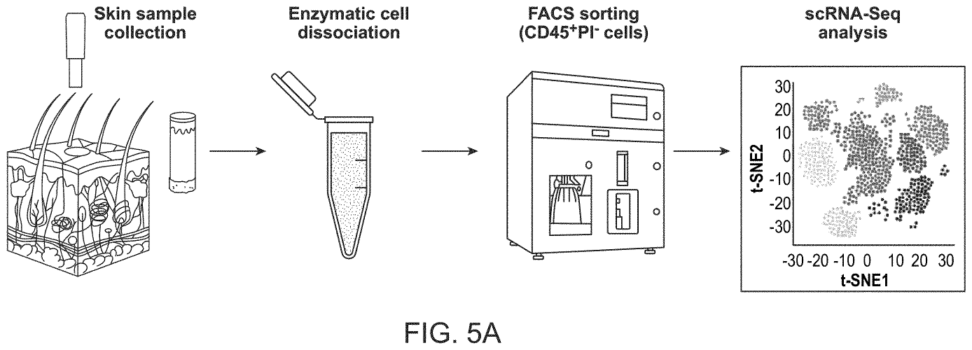

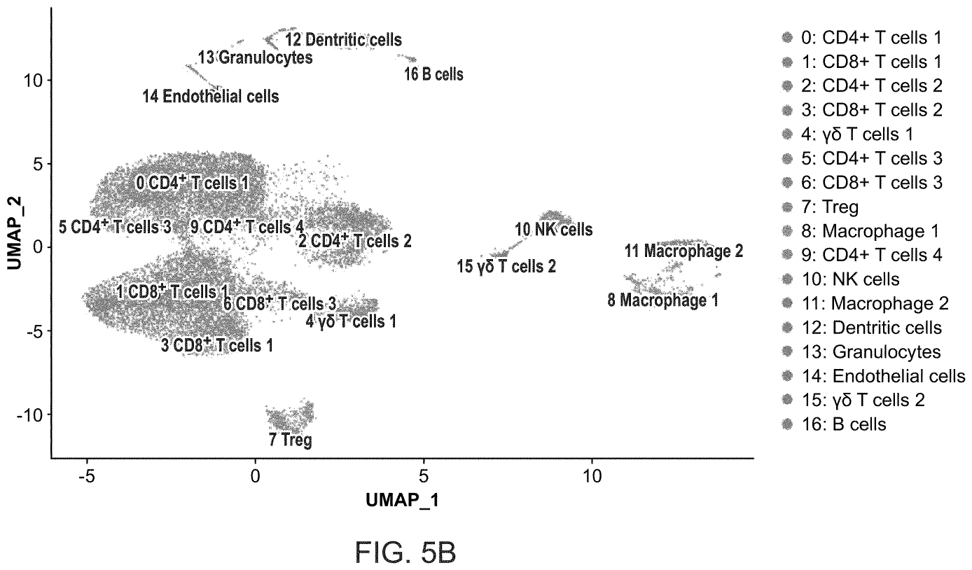

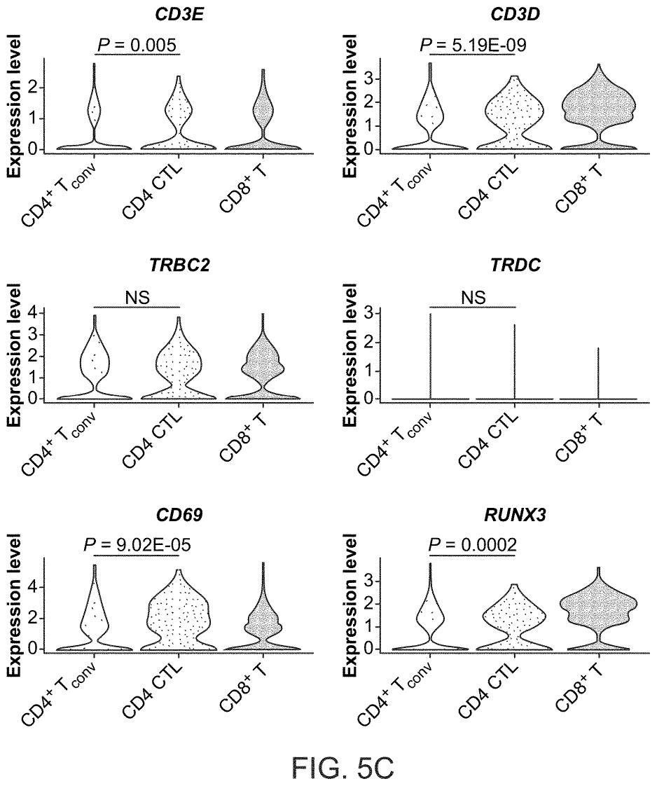

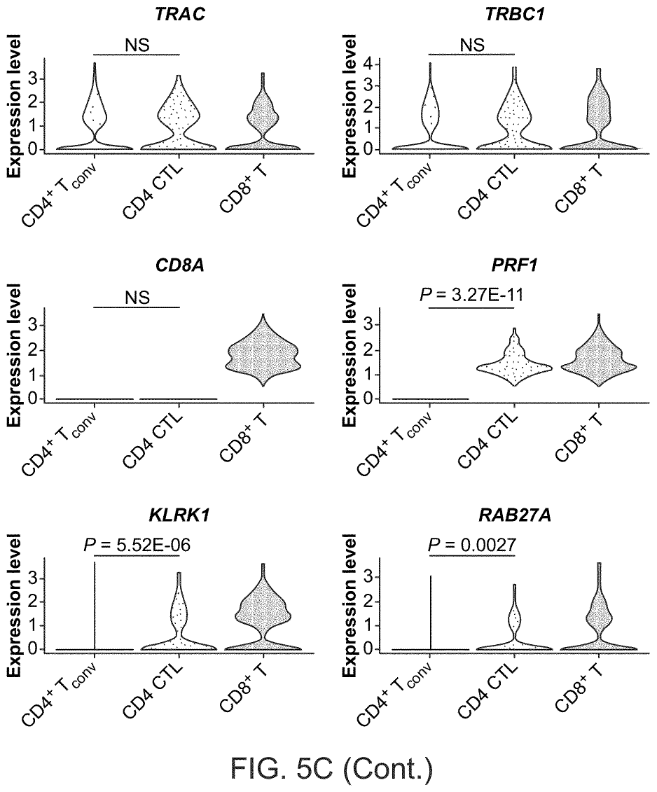

To identify the factor(s) that regulate senescent cells accumulation in the old skin, epidermal condition, blood, or lymphatic vessel density impacted senescent cell numbers were examined. Epidermal thickness, blood and lymphatic vessel density were significantly reduced in the old versus young skin samples; however, these factors had no significant correlation with the number of dermal senescent cells in the old skin samples (FIGS. 3A-3I). A comprehensive skin immune cell profiling using multiplex immunostaining on tissue sections revealed that several innate and adaptive immune cell types were highly enriched in the old dermis (FIG. 4A and FIG. 3J). Importantly, several cytotoxic immune cell types were negatively correlated with the number of dermal senescent cells in the old skin samples (Table 2). Among them, the number of cytotoxic CD4⁺ T cells (CD4 CTLs), which were marked by perforin protein expression, was most negatively correlated (r=−0.6796) with the number of dermal senescent cells in the old skin samples (p<0.0001; FIG. 4B). The number of perforin⁺ CD4 CTLs was significantly increased in the old compared with the young dermis (FIGS. 4C, 4D). Notably, 83.7% of perforin protein-expression dermal cells in the old skin samples were CD4′ T cells (FIG. 4E), indicating that CD4 CTL were the dominant cytotoxic lymphocytes responsible for immunosurveillance in the old human skin. To further characterize CD4 CTLs in the human skin, single-cell RNA sequencing (scRNA-seq) was performed on lymphocyte-enriched single cells from old human skin samples (FIG. 5A). Within the CD4⁺ T cell clusters, a population of perforin (PRF1)-expressing CD4 CTLs, which highly upregulated resident memory marker CD69 (FIGS. 5B and 5C), was identified. In addition, CD4 CTLs expressed cytotoxicity-associated genes, including RUNX3,72,73 RAB27A,74,75 and KLRK1 (encoding for natural killer [NK] group 2D [NKG2D] receptor) (FIG. 5C).76 These findings suggest that CD4 CTL are the effector cells responsible for the clearance of senescent cells in the old human skin.

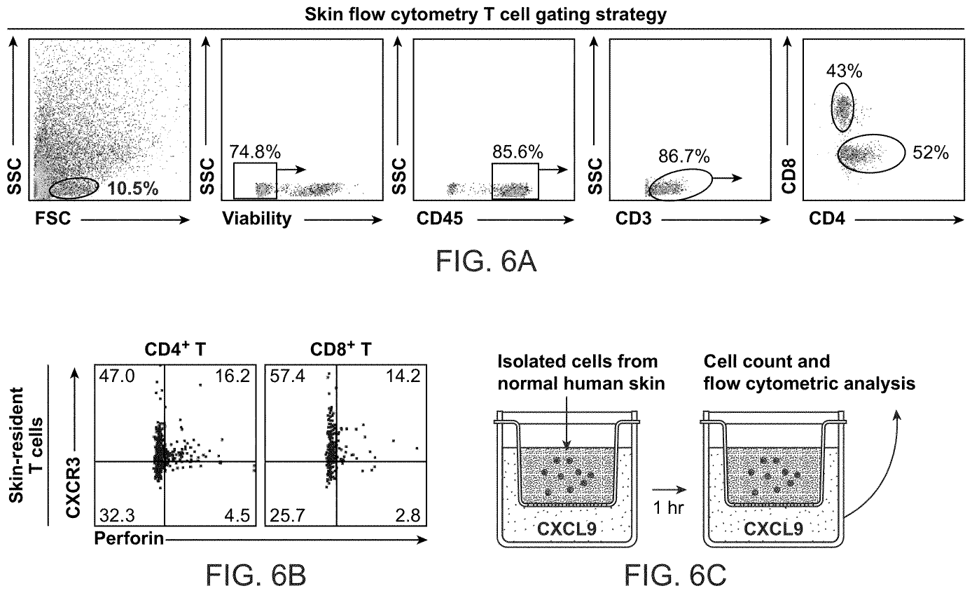

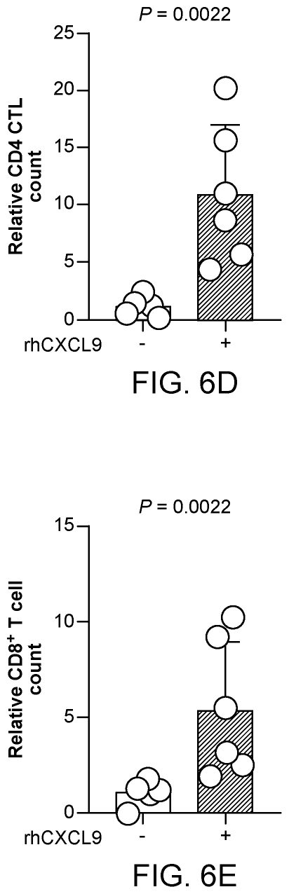

To determine the factors that recruited CD4 CTLs in the old skin, RNA-sequencing (RNA-Seq) of young and old skin samples was performed. Cluster analysis showed that immune-related genes, including CD69, CD96, CD276, CXCL9, KLRD1, IL2RB, IL17RC, IL36G, MB21DI, S100A9 and VTCNI, were among the genes significantly altered in the old versus young skin (FIG. 4F). Among these, CXCL9 chemokine, which is a ligand of CXCR3 expressed on CD4 CTLs19, was focused on. CXCL9 expressing cells were mainly localized in the epidermal basal layer of the old skin samples (FIG. 4G). Dividing the old skin samples into CXCL9^{high} and CXCL9^{low} groups revealed that CXCL9^{high} skin contained a significantly higher number of CD4 CTLs and a lower number of dermal senescent cells compared with CXCL9^{low} skin (FIGS. 4H, I). Furthermore, CD4 CTLs in the human skin expressed CXCR3, and CXCL9 significantly induced the migration of CD4 CTLs in a trans-well assay system (FIG. 6). These findings indicate that CXCL9 expressed by keratinocytes recruits CD4 CTLs to the skin, which may result in the clearance of dermal senescent cells.

TABLE 2
Immune cell correlation with senescent (p16⁺) cell
accumulation in the dermis of the old human skin samples.

| | versus no. of p16⁺ cells in the dermis | |
|---|---|---|
| No. of immune cells in the dermis | r | P value |
| T cell | −0.6619 | <0.0001 *** |
| CD4⁺ T cell | −0.3734 | 0.0421 * |
| CD4 CTL | −0.6796 | <0.0001 *** |
| Th1 | −0.3825 | 0.0369 * |
| Th2 | −0.2823 | 0.1239 |
| Th17 | −0.1772 | 0.3403 |
| Treg | −0.07876 | 0.6736 |
| CD8⁺ T cell | −0.6214 | 0.0002 *** |
| γδ T cell | −0.4674 | 0.0080 ** |
| NKT cell | −0.3417 | 0.0646 |
| cDC1 | 0.3985 | 0.0264 * |
| cDC2 | −0.2004 | 0.2798 |
| LC | −0.2321 | 0.2090 |
| M1 macrophage | −0.4199 | 0.0187 * |
| M2 macrophage | −0.2309 | 0.2115 |
| NK cell | −0.5032 | 0.0090 ** |
| Mast cell | −0.1484 | 0.4256 |
| Neutrophil | −0.4096 | 0.0221 * |

### Example 3. CD4 CTLs Directly Target Senescent Fibroblasts

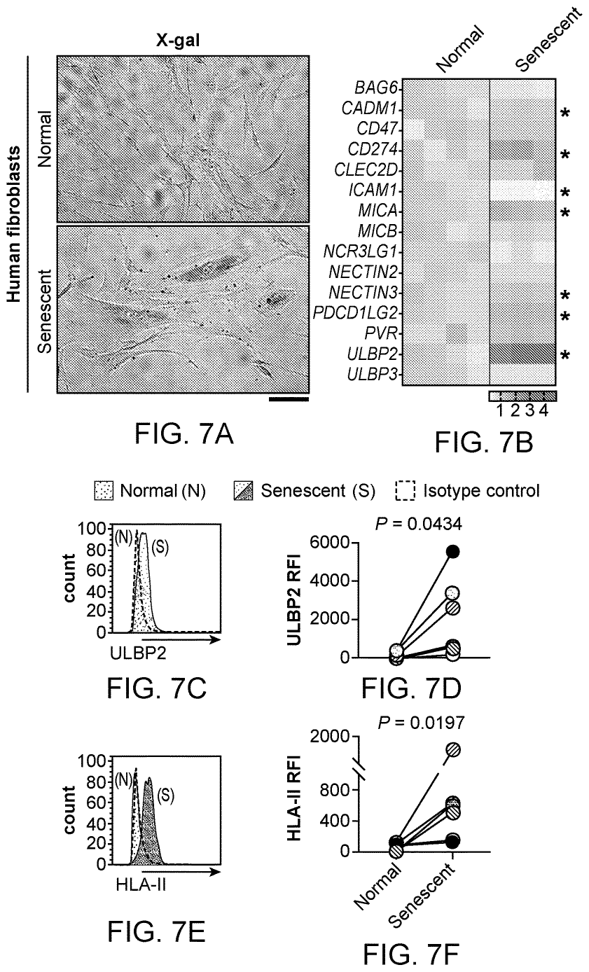

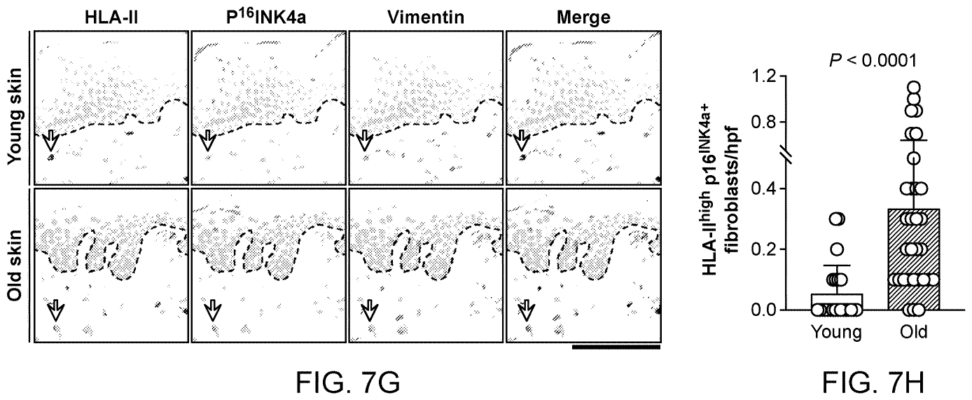

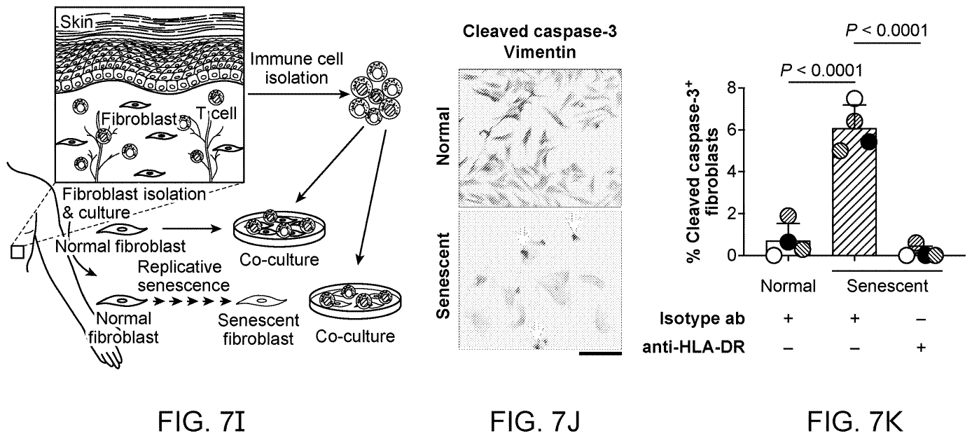

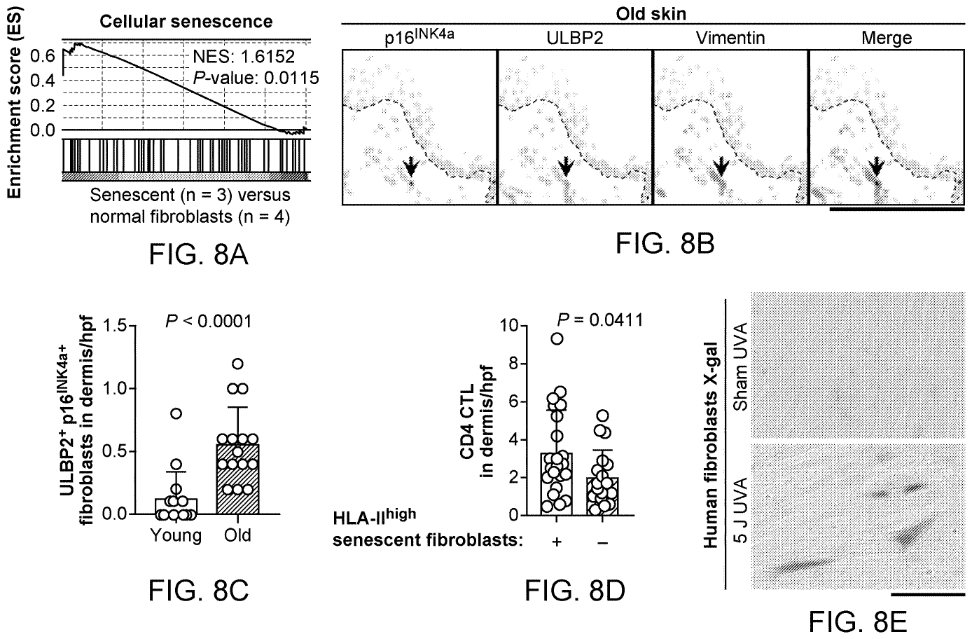

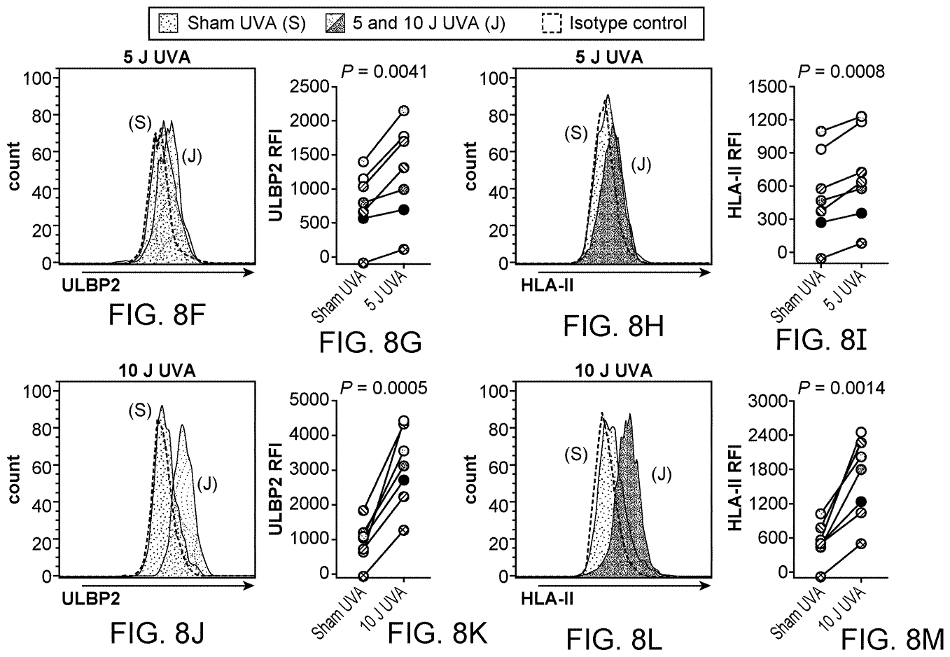

To examine whether CD4 CTLs can target senescent fibroblasts, senescent human dermal fibroblasts were generated ex vivo through repeated passage-induced replicative senescence, which mimics cellular senescence in the sun-protected old skin²⁰ Senescence-associated β-galactosidase (SA-β-Gal) staining marked the senescent fibroblasts (FIG. 7A). RNA-Seq analysis further confirmed the cellular senescence gene set enrichment and revealed that several ligands recognized by cytotoxic lymphocytes were significantly altered in replication-induced senescent compared with normal fibroblasts (FIG. 7B and FIG. 8A). Among these, UL16 Binding Protein 2 (ULBP2), which is a ligand for activating natural killer group 2D (NKG2D) receptor on cytotoxic lymphocytes²¹, was highly upregulated in senescent fibroblasts (FIG. 7B). Accordingly, ULBP2 was expressed on the surface of senescent fibroblasts in contrast to its lack of expression on normal fibroblasts (FIG. 7C, 7D). Likewise, the number of ULBP2″ senescent fibroblasts was significantly increased in the old compared with young human skin (FIGS. 8B and 8C). Human leukocyte antigen class II (HLA-II) surface expression is pivotal for CD4 CTL-elicited immunity. To determine whether senescent fibroblasts could be direct CD4 CTL targets, HLA-II expression on senescent fibroblasts was examined. Importantly, HLA-II was highly expressed on senescent fibroblasts while its expression was negligible on normal fibroblasts (FIG. 7E, 7F). Consistent with this finding, the number of HLA-II^{high} senescent fibroblasts was significantly increased in the old compared with young human skin (FIG. 7G, H). Interestingly, skin samples with a high number of HLA-II^{high} senescent fibroblasts contained a significantly higher number of CD4 CTLs than those without HLA-II^{high} senescent fibroblasts (FIG. 8D). This finding supports the role of the CD4 CTL/HLA-II axis in the immunosurveillance of senescent fibroblasts.

Ultraviolet A (UVA) radiation is a prominent cause of skin aging²², and is known to induce senescence in fibroblasts²². UVA radiation generated senescent human dermal fibroblasts ex vivo (FIG. 8E). ULBP2 and HLA-II levels were markedly increased on the surface of UVA-induced senescent fibroblasts (FIGS. 7A-7M). These findings demonstrate that senescent fibroblasts are highly immunogenic targets, which CD4 CTLs can directly recognize in the human skin.

### Example 4. CD4 CTL Reduce Senescent Fibroblasts in an HLA-II-Dependent Manner

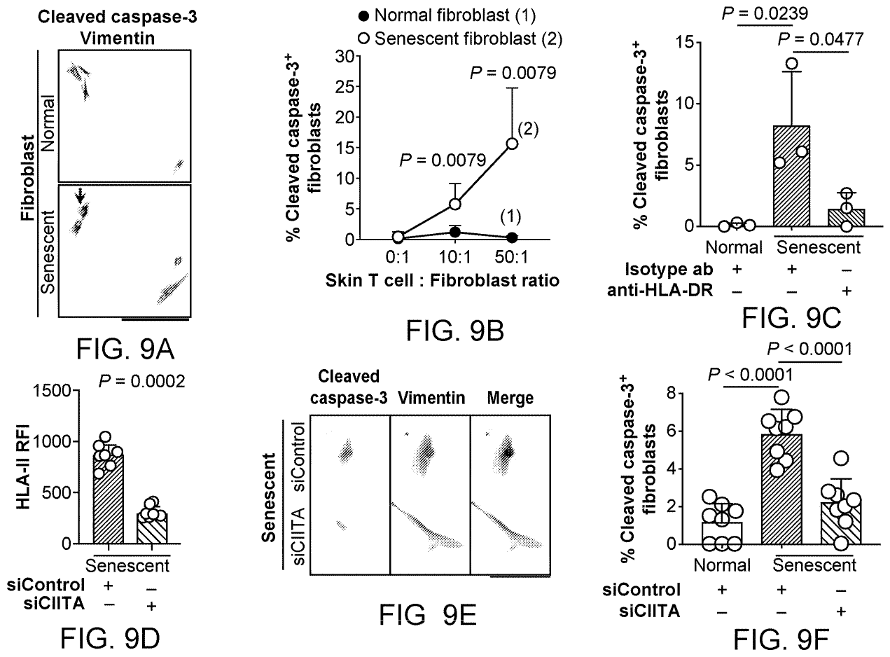

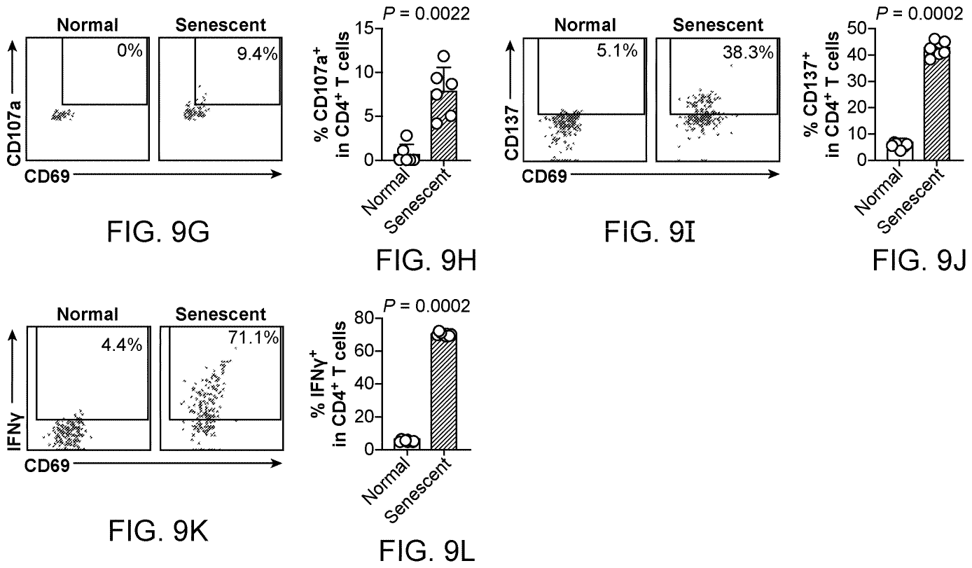

To determine whether CD4 CTL can reduce the level of senescent fibroblasts, normal and replication-induced senescent fibroblasts were co-cultured with immune cells isolated from the old human skin. The skin-derived allogeneic T cells caused apoptosis specifically in the senescent fibroblasts in an HLA-II-dependent manner (FIGS. 9A-9C). Next, a co-culture system was established in which normal and senescent fibroblasts were exposed to autologous T cells that were isolated from the same human skin (FIG. 71). Autologous T cells induced cleaved caspase-3⁺ apoptotic senescent fibroblasts at a significantly higher ratio compared with normal fibroblasts (FIG. 7J, K). Importantly, HLA-DR antibody blockade abolished the skin T cell-induced apoptosis of senescent fibroblasts (FIG. 7K). Furthermore, senescent fibroblasts were generated in which HLA-II was markedly downregulated using small interfering RNA (siRNA) treatment against CIITA, a master regulator of HLA-II gene expression.⁷⁷ HLA-II downregulation significantly blocked the skin immune-cell-induced apoptosis of senescent dermal fibroblasts (FIGS. 9D-9F). Flow-cytometry analysis demonstrated the induction of degranulationmarker, CD107a, and activation markers, CD137 and interferon-g (IFNg), in skin-resident CD4⁺ T cells exposed to senescent compared with normal fibroblasts (FIGS. 9G-9L). These findings demonstrate that senescent fibroblasts are eliminated by T cells in an HLA-II-dependent manner.

### Example 5, CD4 CTL Reduce the Level of Senescent Fibroblasts by Targeting HCMV-gB Antigen

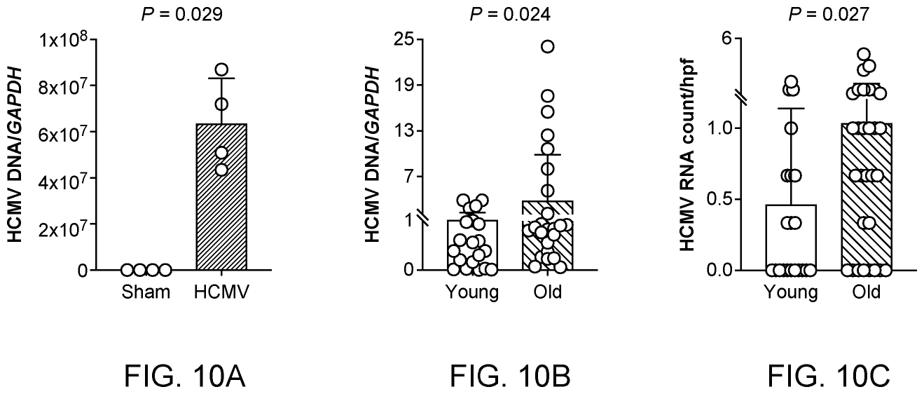

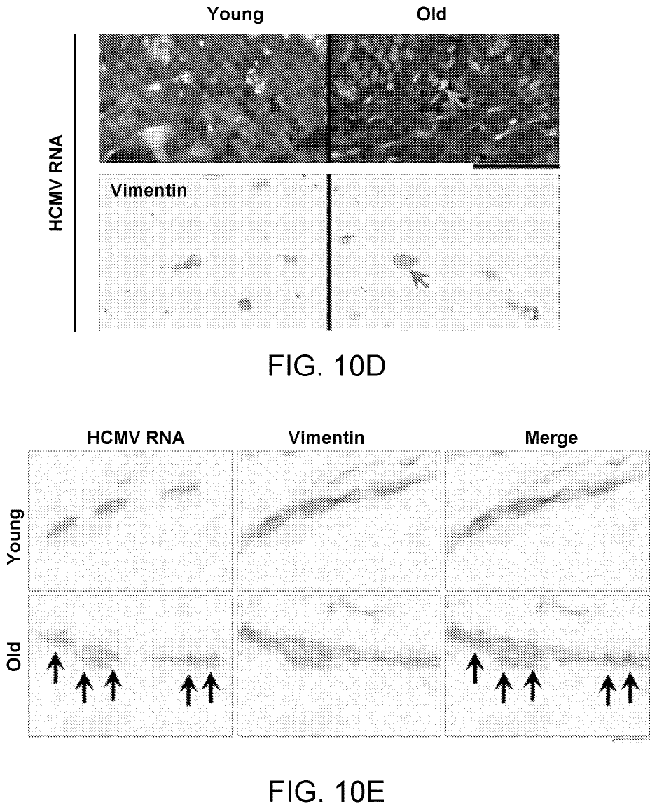

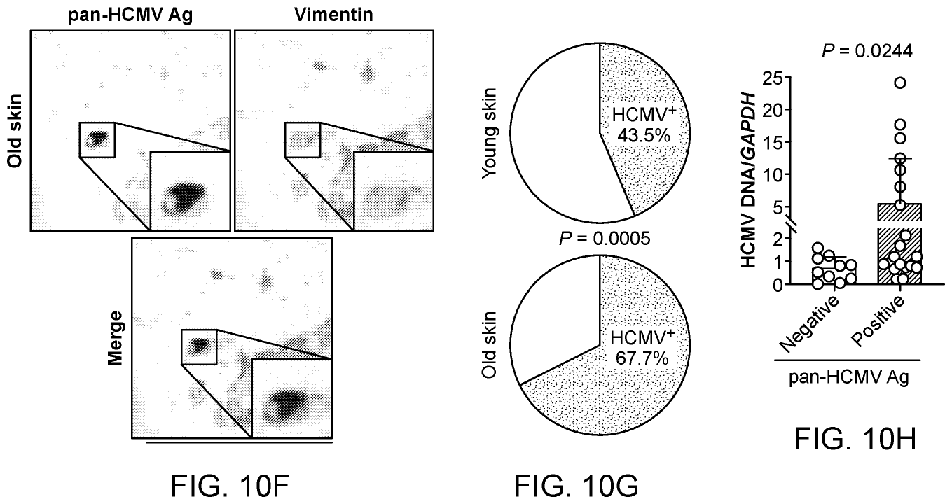

Next, which HLA-II-bound antigen triggered CD4 CTL reduce the level of senescent fibroblasts (e.g., by killing senescent fibroblasts) was investigated. Commensal human cytomegalovirus (HCMV) derived-glycoprotein B (gB) is a principal target of CD4⁺ T cells and can activate CD4 CTL to eliminate host cells in humans²³. Furthermore, gB-specific CD4⁺ T cells are found in the blood of 95% of healthy individuals²⁴. HCMV DNA and RNA were detectable in the normal human skin, and their levels were significantly increased in the old compared with young skin samples (FIGS. 10A-10D). HCMV RNA expression was particularly evident in the dermal fibroblasts in the old skin (FIGS. 10D, E). To further validate the HCMV infection status in the skin samples, a clinical-grade antibody cocktail was used for pan-HCMV antigen detection. HCMV-positive dermal cells were detected in 67.7% of old and 43.5% of young skin samples, which are consistent with seropositivity prevalence in old and young individuals in the United States population (FIGS. 10F and 10G). Notably, pan-HCMV antigen-positive skin samples had higher HCMV DNA levels (FIG. 10H).

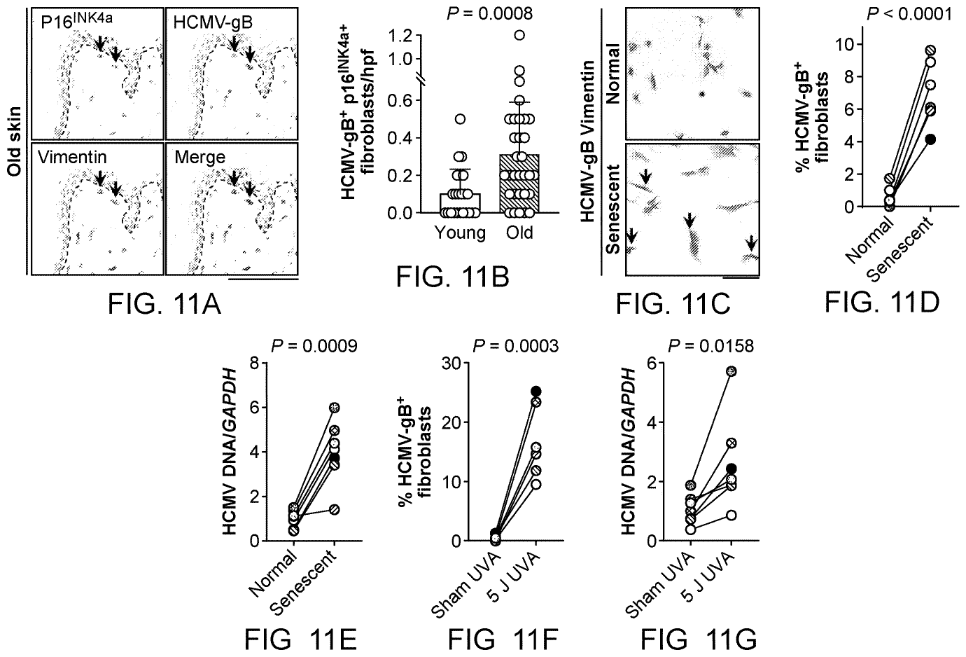

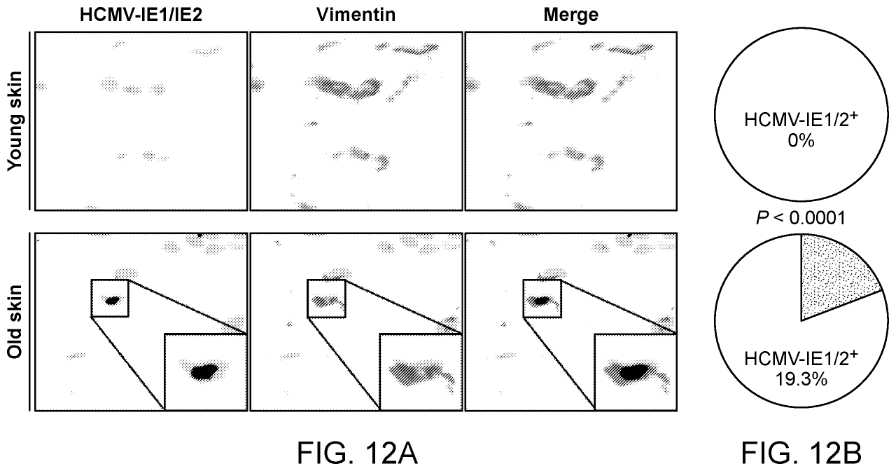

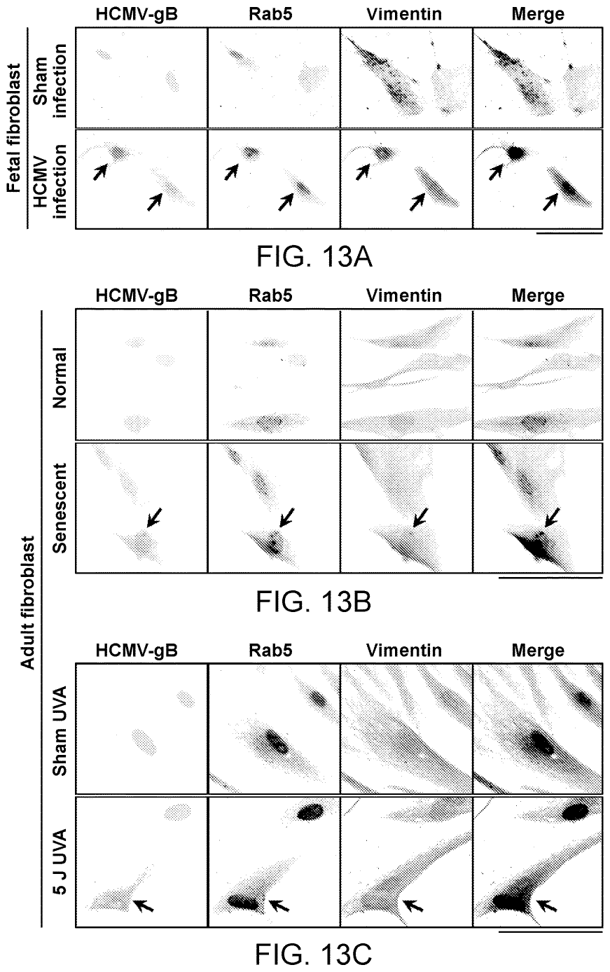

It was found that senescent fibroblasts expressed HCMV-gB in the human skin and the number of HCMV-gB⁺ senescent fibroblasts was significantly increased in the old compared with young skin (FIGS. 11A and 11B). On the other hand, HCMV immediate-early (IE) 1/IE2 proteins were detectable only in a few old skin samples (FIGS. 12A and 12B). In line with the findings in vivo, replication- and UVA-induced senescent fibroblasts highly upregulated HCMV DNA and HCMV-gB expression ex vivo (FIG. 11C-11G). HCMV induces the accumulation of early endosomes in the infected cells^{25,26}, and HCMV-gB is sorted into endosomes and presented on HLA-II²³. Similar to HCMV-infected fetal fibroblasts, HCMV-gB trafficked into endosomes of replication- and UVA-induced adult senescent fibroblasts (FIG. 13). These findings reveal that HCMV is activated in senescent fibroblasts and HCMV-gB can be displayed as an endogenous antigen on HLA-II.

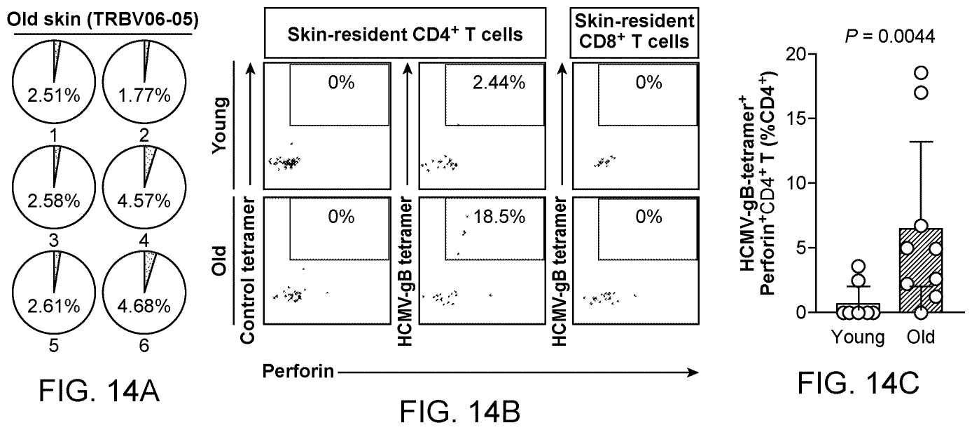

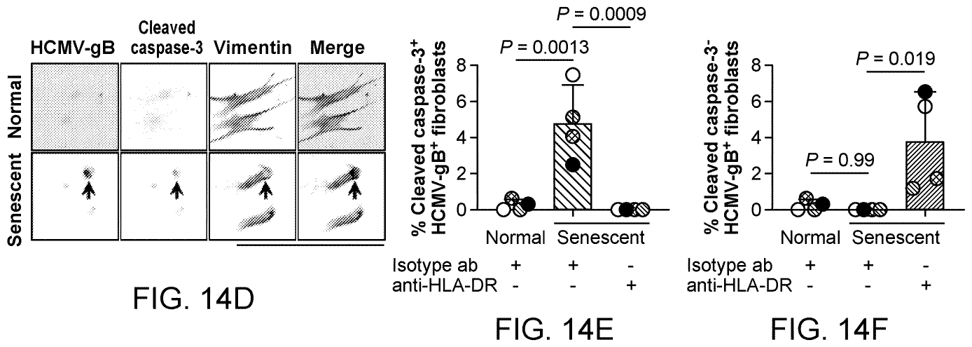

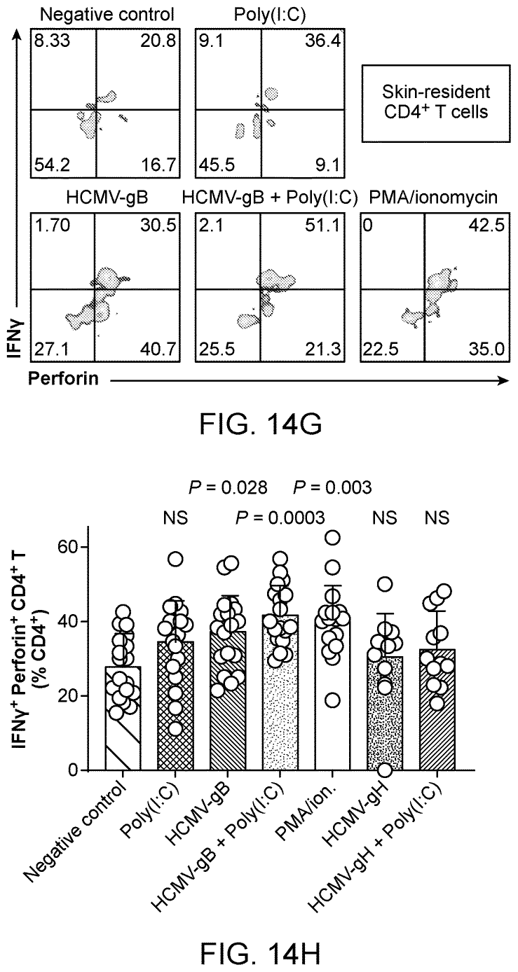

CD4 CTLs that predominantly express T cell receptor (TCR) beta chain TRBV6-5 gene segments recognize HCMV-gB epitopes presented on HLA-DRB1*07:01.^{78,79} Using TCR sequencing on HLA-DRB1*07:01-positive old skin samples, TRBV6-5-expressing T cell clones were detected in the old skin (FIG. 14A). To further determine the presence of HCMV-gB specific CD4 CTLs in the human skin, T cells isolated from young and old human skin was stained with HLA-peptide tetramers that contained the gB-derived DYSNTHSTRYV peptide conjugated to HLA-DRB1*07:01 (DR7).⁸⁰⁻⁸⁴ This analysis demonstrated the presence of HCMV-gB-specific CD4 CTLs in old human skin (FIGS. 14B and 14C).

Autologous T cell/fibroblast co-culture assay revealed that most of the apoptotic senescent fibroblasts expressed HCMV-gB (FIGS. 14D and 14E). Importantly, HCMV-gB⁺ senescent fibroblasts remained intact upon HLA-DR blockade (FIG. 14F). Finally, recombinant HCMV-gB induced IFNγ expression in perforin⁺ CD4 CTLs from the human skin ex vivo (FIGS. 14G and 14H). IFNγ-expressing perforin⁺ CD4CTLs were further increased in the presence of HCMV-gB plus poly (I: C), which is known to enhance the activation of antigen-presenting cells in the skin (FIGS. 14G and 14H).^{86,86} In contrast, HMCV glycoprotein H (gH) plus poly (I: C) did not induce IFNγ expression in CD4 CTLs (FIGS. 14G and 14H). Together, these findings indicate that HCMV-gB-specific CD4 CTLs contribute to the clearance of senescent fibroblasts in aging human skin.

### Example 6. Butyrate and Pentanoate Boost CD4 CTL-Mediated Clearance of Senescent Cells

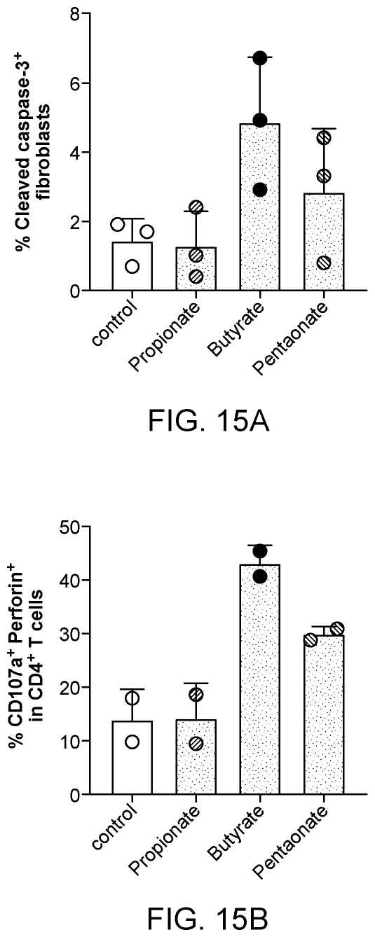

Skin T cells were exposed to 5 mM short chain fatty acids (SCFAs), propionate, butyrate, and pentanoate, for 2 days, and then were co-cultured with senescent dermal fibroblasts. Following 6 h of co-incubation, fibroblasts were stained with vimentin and cleaved caspase-3 to examine the frequency of T cell-mediated cytotoxicity against senescent fibroblasts. T cells exposed to either butyrate or pentanoate, but not propionate, highly induced apoptosis in the senescent cells (FIG. 15A). In addition, skin CD4⁺ T cells exposed to SCFAs, butyrate and pentanoate, showed more cytotoxicity capacity as indicated by an increase in expression of perforin+ and CD107a+ on CD4⁺ T cells (FIG. 15B). These results show that butyrate and pentanoate boost CD4+CTL-mediated clearance of senescent cells.

### REFERENCES

1 He, S. & Sharpless, N. E. Senescence in Health and Disease. Cell 169, 1000-1011, doi:10.1016/j.cell.2017.05.015 (2017).
2 Di Micco, R., Krizhanovsky, V., Baker, D. & d′Adda di Fagagna, F. Cellular senescence in ageing: from mechanisms to therapeutic opportunities. Nat Rev Mol Cell Biol 22, 75-95, doi:10.1038/s41580-020-00314-w (2021).
3 Mahmoudi, S., Xu, L. & Brunet, A. Turning back time with emerging rejuvenation strategies. Nat Cell Biol 21, 32-43, doi:10.1038/s41556-018-0206-0 (2019).
4 Kirkland, J. L. & Tchkonia, T. Cellular Senescence: A Translational Perspective. EBioMedicine 21, 21-28, doi:10.1016/j.ebiom.2017.04.013 (2017).
5 Paez-Ribes, M., Gonzalez-Gualda, E., Doherty, G. J. & Munoz-Espin, D. Targeting senescent cells in translational medicine. EMBO Mol Med 11, e10234, doi:10.15252/emmm.201810234 (2019).
6 Krizhanovsky, V. et al. Senescence of activated stellate cells limits liver fibrosis. Cell 134, 657-667, doi:10.1016/j.cell.2008.06.049 (2008).
7 Kang, T. W. et al. Senescence surveillance of pre-malignant hepatocytes limits liver cancer development. Nature 479, 547-551, doi:10.1038/nature10599 (2011).
8 Kansara, M. et al. Immune response to RB1-regulated senescence limits radiation-induced osteosarcoma formation. J Clin Invest 123, 5351-5360, doi:10.1172/JCI70559 (2013).
9 Pereira, B. I. et al. Senescent cells evade immune clearance via HLA-E-mediated NK and CD8 (+) T cell inhibition. Nature communications 10, 2387, doi:10.1038/s41467-019-10335-5 (2019).
10 Wright, W. E. & Shay, J. W. Telomere dynamics in cancer progression and prevention: fundamental differences in human and mouse telomere biology. Nat Med 6, 849-851, doi:10.1038/78592 (2000).
11 Wadhwa, R. et al. A major functional difference between the mouse and human ARF tumor suppressor proteins. J Biol Chem 277, 36665-36670, doi:10.1074/jbc.M203222200 (2002).
12 Parrinello, S. et al. Oxygen sensitivity severely limits the replicative lifespan of murine fibroblasts. Nat Cell Biol 5, 741-747, doi:10.1038/ncb1024 (2003).
13 Itahana, K., Campisi, J. & Dimri, G. P. Mechanisms of cellular senescence in human and mouse cells. Biogerontology 5, 1-10, doi:10.1023/b: bgen.0000017682.96395.10 (2004).
14 Coppe, J. P. et al. A human-like senescence-associated secretory phenotype is conserved in mouse cells dependent on physiological oxygen. PLOS One 5, e9188, doi:10.1371/journal.pone. 0009188 (2010).
15 Mestas, J. & Hughes, C. C. Of mice and not men: differences between mouse and human immunology. J Immunol 172, 2731-2738, doi:10.4049/jimmunol. 172.5.2731 (2004).
16 Carr, E. J. et al. The cellular composition of the human immune system is shaped by age and cohabitation. Nat Immunol 17, 461-468, doi:10.1038/ni.3371 (2016).
17 Pulendran, B. & Davis, M. M. The science and medicine of human immunology. Science 369, doi:10.1126/science.aay4014 (2020).
18 Ressler, S. et al. p16INK4A is a robust in vivo biomarker of cellular aging in human skin. Aging Cell 5, 379-389, doi:10.1111/j.1474-9726.2006.00231.x (2006).
19 Takeuchi, A. et al. CRTAM determines the CD4⁺ cytotoxic T lymphocyte lineage. J Exp Med 213, 123-138, doi:10.1084/jem.20150519 (2016).
20 Campisi, J. The role of cellular senescence in skin aging. J Investig Dermatol Symp Proc 3, 1-5 (1998).
21 Gonzalez, S., Lopez-Soto, A., Suarez-Alvarez, B., Lopez-Vazquez, A. & Lopez-Larrea, C. NKG2D ligands: key targets of the immune response. Trends Immunol 29, 397-403, doi:10.1016/j.it.2008.04.007 (2008).
22 Lan, C. E., Hung, Y. T., Fang, A. H. & Ching-Shuang, W. Effects of irradiance on UVA-induced skin aging. J Dermatol Sci 94, 220-228, doi:10.1016/j.jdermsci.2019.03.005 (2019).
23 Hegde, N. R. et al. Endogenous human cytomegalovirus gB is presented efficiently by MHC class II molecules to CD4+CTL. The Journal of experimental medicine 202, 1109-1119, doi:10.1084/jem.20050162 (2005).
24 Pachnio, A., Zuo, J., Ryan, G. B., Begum, J. & Moss, P. A. The Cellular Localization of Human Cytomegalovirus Glycoprotein Expression Greatly Influences the Frequency and Functional Phenotype of Specific CD4⁺ T Cell Responses. J Immunol 195, 3803-3815, doi:10.4049/jimmunol.1500696 (2015).
25 Lucin, P. et al. Cytomegalovirus Generates Assembly Compartment in the Early Phase of Infection by Perturbation of Host-Cell Factors Recruitment at the Early Endosome/Endosomal Recycling Compartment/Trans-Golgi Interface. Front Cell Dev Biol 8, 563607, doi:10.3389/fcell.2020.563607 (2020).
26 Karleusa, L., Mahmutefendic, H., Tomas, M. I., Zagorac, G. B. & Lucin, P. Landmarks of endosomal remodeling in the early phase of cytomegalovirus infection. Virology 515, 108-122, doi:10.1016/j.virol.2017.12.001 (2018).
27 Takeuchi, A. & Saito, T. CD4 CTL, a Cytotoxic Subset of CD4 (+) T Cells, Their Differentiation and Function. Front Immunol 8, 194, doi:10.3389/fimmu.2017.00194 (2017).
28 Oh, D. Y. & Fong, L. Cytotoxic CD4 (+) T cells in cancer: Expanding the immune effector toolbox. Immunity 54, 2701-2711, doi:10.1016/j.immuni.2021.11.015 (2021).
29 Hashimoto, K. et al. Single-cell transcriptomics reveals expansion of cytotoxic CD4 T cells in supercentenarians. Proceedings of the National Academy of Sciences of the United States of America 116, 24242-24251, doi:10.1073/pnas. 1907883116 (2019).
30 Parry, H. M. et al. PD-1 is imprinted on cytomegalovirus-specific CD4⁺ T cells and attenuates Th1 cytokine production whilst maintaining cytotoxicity. PLOS Pathog 17, e1009349, doi:10.1371/journal.ppat. 1009349 (2021).
31 Noris, E. et al. Cell cycle arrest by human cytomegalovirus 86-kDa IE2 protein resembles premature senescence. Journal of virology 76, 12135-12148, doi:10.1128/jvi.76.23.12135-12148.2002 (2002).
32 Watanabe, R. et al. Human skin is protected by four functionally and phenotypically discrete populations of resident and recirculating memory T cells. Science translational medicine 7, 279ra239, doi:10.1126/scitranslmed.3010302 (2015).
33 Picelli, S. et al. Full-length RNA-seq from single cells using Smart-seq2. Nat Protoc 9, 171-181, doi:10.1038/nprot.2014.006 (2014).
34 Dobin, A. et al. STAR: ultrafast universal RNA-seq aligner. Bioinformatics 29, 15-21, doi:10.1093/bioinformatics/bts635 (2013).
35 Li, B. & Dewey, C. N. RSEM: accurate transcript quantification from RNA-Seq data with or without a reference genome. BMC Bioinformatics 12, 323, doi:10.1186/1471-2105-12-323 (2011).
36 Love, M. I., Huber, W. & Anders, S. Moderated estimation of fold change and dispersion for RNA-seq data with DESeq2. Genome Biol 15, 550, doi:10.1186/s13059-014-0550-8 (2014).
37 Schiferle, E. B. et al. Rejection of benign melanocytic nevi by nevus-resident CD4 (+) T cells. Sci Adv 7, doi:10.1126/sciadv.abg4498 (2021).
38 Kasztelewicz, B. et al. Cytokine gene polymorphism associations with congenital cytomegalovirus infection and sensorineural hearing loss. Eur J Clin Microbiol Infect Dis 36, 1811-1818, doi:10.1007/s10096-017-2996-6 (2017).
39 Czech-Kowalska, J. et al. The Limitations of Cytomegalovirus DNA Detection in Cerebrospinal Fluid of Newborn Infants With Congenital CMV Infection: A Tertiary Care Neonatal Center Experience. Pediatr Infect Dis J 40, 838-845, doi:10.1097/INF.0000000000003205 (2021).
40 Trapani, J. A., and Smyth, M. J. (2002). Functional significance of the perforin/granzyme cell death pathway. Nat. Rev. Immunol. 2, 735-747. doi.org/10.1038/nri911.
41 Hegde, N. R., Dunn, C., Lewinsohn, D. M., Jarvis, M. A., Nelson, J. A., and Johnson, D. C. (2005). Endogenous human cytomegalovirus gB is presented efficiently by MHC class II molecules to CD4+CTL. J. Exp. Med. 202, 1109-1119. doi.org/10.1084/jem.20050162.
42 Rawlinson, W. D., et al. (2017). Congenital cytomegalovirus infection in pregnancy and the neonate: consensus recommendations for prevention, diagnosis, and therapy. Lancet Infect. Dis. 17, e177-e188. doi.org/10.1016/S1473-3099 (17) 30143-3.
43 Griffiths, P., and Reeves, M. (2021). Pathogenesis of human cytomegalovirus in the immunocompromised host. Nat. Rev. Microbiol. 19, 759-773. doi.org/10.1038/s41579-021-00582-z.
44 Shenk, T., and Alwine, J. C. (2014). Human cytomegalovirus: coordinating cellular stress, signaling, and metabolic pathways. Annu. Rev. Virol. 1, 355-374. doi.org/10.1146/annurev-virology-031413-085425.
45 Brodin, P., Jojic, V., Gao, T., Bhattacharya, S., Angel, C. J., Furman, D., Shen-Orr, S., Dekker, C. L., Swan, G. E., Butte, A. J., et al. (2015). Variation in the human immune system is largely driven by non-heritable influences. Cell 160, 37-47. doi.org/10.1016/j.cell.2014.12.020.
46 Hein, M. Y., and Weissman, J. S. (2022). Functional single-cell genomics of human cytomegalovirus infection. Nat. Biotechnol. 40, 391-401. doi.org/10.1038/s41587-021-01059-3.
47 Sinzger, C., Grefte, A., Plachter, B., Gouw, A. S., The, T. H., and Jahn, G. (1995). Fibroblasts, epithelial cells, endothelial cells and smooth muscle cells are major targets of human cytomegalovirus infection in lung and gastrointestinal tissues. J. Gen. Virol. 76, 741-750. doi.org/10.1099/0022-1317-76-4-741.
48 Krizhanovsky, V., Yon, M., Dickins, R. A., Hearn, S., Simon, J., Miething, C., Yee, H., Zender, L., and Lowe, S. W. (2008). Senescence of activated stellate cells limits liver fibrosis. Cell 134, 657-667. doi.org/10.1016/j. cell.2008.06.049.
49 Kang, T. W., Yevsa, T., Woller, N., Hoenicke, L., Wuestefeld, T., Dauch, D., Hohmeyer, A., Gereke, M., Rudalska, R., Potapova, A., et al. (2011). Senescence surveillance of pre-malignant hepatocytes limits liver cancer development. Nature 479, 547-551.
50 Wang, T. W., Johmura, Y., Suzuki, N., Omori, S., Migita, T., Yamaguchi, K., Hatakeyama, S., Yamazaki, S., Shimizu, E., Imoto, S., et al. (2022). Blocking PD-L1-PD-1 improves senescence surveillance and ageing phenotypes. Nature 611, 358-364. doi.org/10.1038/s41586-022-05388-4.
51 Marin, I., Boix, O., Garcia-Garijo, A., Sirois, I., Caballe, A., Zarzuela, E., Ruano, I., Attolini, C. S., Prats, N., López-Domínguez, J. A., et al. (2023). Cellular senescence is immunogenic and promotes anti-tumor immunity. Cancer Discov. 13, 410-431. doi.org/10.1158/2159-8290.CD-22-0523.
52 Chen, H. A., Ho, Y. J., Mezzadra, R., Adrover, J. M., Smolkin, R., Zhu, C., Woess, K., Bernstein, N., Schmitt, G., Fong, L., et al. (2023). Senescence rewires microenvironment sensing to facilitate anti-tumor immunity. Cancer Discov. 13, 432-453. doi.org/10.1158/2159-8290.CD-22-0528.
53 Gilioli, D., Fusco, S., Tavella, T., Giannetti, K., Conti, A., Santoro, A., Carsana, E., Beretta, S., Schönlein, M., Gambacorta, V., et al. (2022). Therapy-induced senescence upregulates antigen presentation machinery and triggers anti-tumor immunity in Acute myeloid leukemia. Preprint at bioRxiv. doi.org/10.1101/2022.11.17.515658.
54 Appay, V., Zaunders, J. J., Papagno, L., Sutton, J., Jaramillo, A., Waters, A., Easterbrook, P., Grey, P., Smith, D., McMichael, A. J., et al. (2002). Characterization of CD4 (+) CTLs ex vivo. J. Immunol. 168, 5954-5958. doi.org/10.4049/jimmunol.168.11.5954.
55 Khanna, R., Burrows, S. R., Thomson, S. A., Moss, D. J., Cresswell, P., Poulsen, L. M., and Cooper, L. (1997). Class I processing-defective Burkitt's lymphoma cells are recognized efficiently by CD4+EBV-specific CTLs. J. Immunol. 158, 3619-3625.
56 Baz-Martínez, M., Da Silva-Alvarez, S., Rodríguez, E., Guerra, J., El Motiam, A., Vidal, A., García-Caballero, T., González-Barcia, M., Sánchez, L., Muñoz-Fontela, C., et al. (2016). Cell senescence is an antiviral defense mechanism. Sci. Rep. 6, 37007. doi.org/10.1038/srep37007.
57 Reddel, R. R. (2010). Senescence: an antiviral defense that is tumor suppressive? Carcinogenesis 31, 19-26. doi.org/10.1093/carcin/bgp274.
58 Pachnio, A., Zuo, J., Ryan, G. B., Begum, J., and Moss, P. A. (2015). The cellular localization of human cytomegalovirus glycoprotein expression greatly influences the frequency and functional phenotype of specific CD4⁺ T cell responses. J. Immunol. 195, 3803-3815. doi.org/10.4049/jimmunol. 1500696.
59 Cannon, M. J., Schmid, D. S., and Hyde, T. B. (2010). Review of cytomegalovirus seroprevalence and demographic characteristics associated with infection. Rev. Med. Virol. 20, 202-213. doi.org/10.1002/rmv.655.
60 Satija, R., Farrell, J. A., Gennert, D., Schier, A. F., and Regev, A. (2015). Spatial reconstruction of single-cell gene expression data. Nat. Biotechnol. 33, 495-502. doi.org/10.1038/nbt.3192.
61 Wanjalla, C. N., McDonnell, W. J., Ram, R., Chopra, A., Gangula, R., Leary, S., Mashayekhi, M., Simmons, J. D., Warren, C. M., Bailin, S., et al. (2021). Single-cell analysis shows that adipose tissue of persons with both HIV and diabetes is enriched for clonal, cytotoxic, and CMV-specific CD4⁺ T cells. Cell Rep. Med. 2, 100205. doi.org/10.1016/j.xcrm.2021.100205.
62 Parry, H. M., Dowell, A. C., Zuo, J., Verma, K., Kinsella, F. A. M., Begum, J., Croft, W., Sharma-Oates, A., Pratt, G., and Moss, P. (2021). PD-1 is imprinted on cytomegalovirus-specific CD4⁺ T cells and attenuates Th1 cytokine production whilst maintaining cytotoxicity. PLOS Pathog. 17, e1009349. doi.org/10.1371/journal.ppat. 1009349.
63 Oja, A. E., Vieira Braga, F. A., Remmerswaal, E. B., Kragten, N. A., Hertoghs, K. M., Zuo, J., Moss, P. A., van Lier, R. A., van Gisbergen, K. P., and Hombrink, P. (2017). The transcription factor hobit identifies human cytotoxic CD4 (+) T cells. Front. Immunol. 8, 325. doi.org/10.3389/fimmu.2017.00325.
64 Abana, C. O., Pilkinton, M. A., Gaudieri, S., Chopra, A., McDonnell, W. J., Wanjalla, C., Barnett, L., Gangula, R., Hager, C., Jung, D. K., et al. (2017). Cytomegalovirus (CMV) epitope-specific CD4 (+) T cells are inflated in HIV (+) CMV (+) subjects. J. Immunol. 199, 3187-3201. doi.org/10.4049/jimmunol.1700851.
65 Pachnio, A., Ciaurriz, M., Begum, J., Lal, N., Zuo, J., Beggs, A., and Moss, P. (2016). Cytomegalovirus infection leads to development of high frequencies of cytotoxic virus-specific CD4⁺ T cells targeted to vascular endothelium. PLOS Pathog. 12, e1005832. doi.org/10.1371/journal.ppat.1005832.
66 Elkington, R., Shoukry, N. H., Walker, S., Crough, T., Fazou, C., Kaur, A., Walker, C. M., and Khanna, R. (2004). Cross-reactive recognition of human and primate cytomegalovirus sequences by human CD4 cytotoxic T lymphocytes specific for glycoprotein B and H. Eur. J. Immunol. 34, 3216-3226. doi.org/10.1002/eji.200425203.
67 Stein, G. H., Drullinger, L. F., Soulard, A., and Dulić, V. (1999). Differential roles for cyclin-dependent kinase inhibitors p21 and p16 in the mechanisms of senescence and differentiation in human fibroblasts. Mol. Cell. Biol. 19, 2109-2117. doi.org/10.1128/MCB.19.3.2109.
68 Chandra, A., Lagnado, A. B., Farr, J. N., Doolittle, M., Tchkonia, T., Kirkland, J. L., LeBrasseur, N. K., Robbins, P. D., Niedernhofer, L. J., Ikeno, Y., et al. (2022). Targeted clearance of p21- but not p16-positive senescent cells prevents radiation-induced osteoporosis and increased marrow adiposity. Aging Cell 21, e13602. doi.org/10.1111/acel.13602.
69 López-Domínguez, J. A., et al. (2021). Cdknla transcript variant 2 is a marker of aging and cellular senescence. Aging (Albany, N. Y.) 13, 13380-13392.doi.org/10.18632/aging.203110.
70 Chandra, A., Lagnado, A. B., Farr, J. N., Monroe, D. G., Park, S., Hachfeld, C., Tchkonia, T., Kirkland, J. L., Khosla, S., Passos, J. F., and Pignolo, R. J. (2020). Targeted reduction of senescent cell burden alleviates focal radiotherapy-related bone loss. J. Bone Miner. Res. 35, 1119-1131. doi.org/10.1002/jbmr.3978.
71 Evangelou, K., et al. (2017). Robust, universal biomarker assay to detect senescent cells in biological specimens. Aging Cell 16, 192-197. doi.org/10.1111/acel.12545.
72 Knudson, C. J., et al. (2021). Mechanisms of antiviral cytotoxic CD4 T cell differentiation. J. Virol. 95, e0056621. doi.org/10.1128/JVI.00566-21.
73 Cenerenti, M., Saillard, M., Romero, P., and Jandus, C. (2022). The era of cytotoxic CD4 T cells. Front. Immunol. 13, 867189. doi.org/10. 3389/fimmu.2022.867189.
74 Fukuda, M. (2013). Rab27 effectors, pleiotropic regulators in secretory pathways. Traffic 14, 949-963. doi.org/10.1111/tra.12083.
75 Stinchcombe, J. C., Barral, D. C., Mules, E. H., Booth, S., Hume, A. N., Machesky, L. M., Seabra, M. C., and Griffiths, G. M. (2001). Rab27a is required for regulated secretion in cytotoxic T lymphocytes. J. Cell Biol. 152, 825-834. doi.org/10.1083/jcb. 152.4.825.
76 González, S., et al. (2008). NKG2D ligands: key targets of the immune response. Trends Immunol. 29, 397-403. doi.org/10.1016/j.it. 2008.04.007.
77 Ting, J. P., and Trowsdale, J. (2002). Genetic control of MHC class II expression. Cell 109, S21-S33. doi.org/10.1016/s0092-8674 (02) 00696-7.
78 Elkington, R., Shoukry, N. H., Walker, S., Crough, T., Fazou, C., Kaur, A., Walker, C. M., and Khanna, R. (2004). Cross-reactive recognition of human and primate cytomegalovirus sequences by human CD4 cytotoxic T lymphocytes specific for glycoprotein B and H. Eur. J. Immunol. 34, 3216-3226. doi.org/10.1002/eji.200425203.
79 Crompton, L., Khan, N., Khanna, R., Nayak, L., and Moss, P. A. (2008).CD4⁺ T cells specific for glycoprotein B from cytomegalovirus exhibit extreme conservation of T-cell receptor usage between different individuals. Blood 111, 2053-2061. doi.org/10.1182/blood-2007-04-079863.
80 Wanjalla, C. N., McDonnell, W. J., Ram, R., Chopra, A., Gangula, R., Leary, S., Mashayekhi, M., Simmons, J. D., Warren, C. M., Bailin, S., et al. (2021). Single-cell analysis shows that adipose tissue of persons with both HIV and diabetes is enriched for clonal, cytotoxic, and CMV-specific CD4⁺ T cells. Cell Rep. Med. 2, 100205. doi.org/10.1016/j.xcrm.2021. 100205.
81 Parry, H. M., Dowell, A. C., Zuo, J., Verma, K., Kinsella, F. A. M., Begum, J., Croft, W., Sharma-Oates, A., Pratt, G., and Moss, P. (2021). PD-1 is imprinted on cytomegalovirus-specific CD4⁺ T cells and attenuates Th1 cytokine production whilst maintaining cytotoxicity. PLOS Pathog. 17,e1009349. doi.org/10.1371/journal.ppat. 1009349.
82 Oja, A. E., Vieira Braga, F. A., Remmerswaal, E. B., Kragten, N. A., Hertoghs, K. M., Zuo, J., Moss, P. A., van Lier, R. A., van Gisbergen, K. P., and Hombrink, P. (2017). The transcription factor hobit identifies human cytotoxic CD4 (+) T cells. Front. Immunol. 8, 325. doi.org/10.3389/fimmu.2017.00325.
83 Abana, C. O., Pilkinton, M. A., Gaudieri, S., Chopra, A., McDonnell, W. J., Wanjalla, C., Barnett, L., Gangula, R., Hager, C., Jung, D. K., et al. (2017). Cytomegalovirus (CMV) epitope-specific CD4 (+) T cells are inflated in HIV (+) CMV (+) subjects. J. Immunol. 199, 3187-3201. doi.org/10.4049/jimmunol. 1700851.
84 Pachnio, A., Ciaurriz, M., Begum, J., Lal, N., Zuo, J., Beggs, A., and Moss, P. (2016). Cytomegalovirus infection leads to development of high frequencies of cytotoxic virus-specific CD4⁺ T cells targeted to vascular endothelium. PLOS Pathog. 12, e1005832. doi.org/10.1371/journal.ppat. 1005832.
85 Apostólico, J. S. (2019). Poly (I: C) potentiates T cell immunity to a dendritic cell targeted HIV-multiepitope vaccine. Front. Immunol. 10, 843. doi.org/10.3389/fimmu.2019.00843.
86 Quinn, K. M., Yamamoto, A., Costa, A., Darrah, P. A., Lindsay, R. W., Hegde, S. T., Johnson, T. R., Flynn, B. J., Loré, K., and Seder, R. A. (2013). Coadministration of polyinosinic: polycytidylic acid and immunostimulatory complexes modifies antigen processing in dendritic cell subsets and enhances HIV gag-specific T cell immunity. J. Immunol. 191, 5085-5096. doi.org/10.4049/jimmunol.1301730.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of delaying the onset or slowing progression of aging in a subject, the method comprising administering to the subject an effective amount of a composition comprising:
a plurality of (i) antigenic proteins from commensal human cytomegaloviruses (HCMV), (ii) antigenic peptides derived from proteins from commensal human cytomegaloviruses, or (iii) live or live-attenuated commensal human cytomegaloviruses; and
a T cell adjuvant that increases T cell response to the plurality of the antigenic proteins, the antigenic peptides, or the live or live-attenuated commensal human cytomegaloviruses.

2. A method of reducing the level of senescent fibroblasts in a subject, the method comprising administering to the subject an effective amount of a composition comprising:
a plurality of (i) antigenic proteins from commensal human cytomegaloviruses (HCMV), (ii) antigenic peptides derived from proteins from commensal human cytomegaloviruses, or (iii) live or live-attenuated commensal human cytomegaloviruses; and
a T cell adjuvant that increases T cell response to the plurality of the antigenic proteins, the antigenic peptides, or the live or live-attenuated commensal human cytomegaloviruses.

3. The method of claim 2, wherein the level of senescent fibroblasts is reduced in the skin of the subject.

4. The method of any one of claims 1-3, wherein the subject has an aging-associated disease.

5. A method of treating, or reducing the risk of developing, an aging-associated disease or condition in a subject, the method comprising administering to the subject an effective amount of a composition comprising:
a plurality of (i) antigenic proteins from commensal human cytomegaloviruses (HCMV), (ii) antigenic peptides derived from proteins from commensal human cytomegaloviruses, or (iii) live or live-attenuated commensal human cytomegaloviruses; and
a T cell adjuvant that increases T cell response to plurality of the antigenic proteins, the antigenic peptides, or the live or live-attenuated commensal human cytomegaloviruses.

6. The method of claim 4 or claim 5, wherein the aging-associated disease is one or more of a cancer, a cardiovascular disease, a neurodegenerative disease, a renal disease, an autoimmune disease, arthritis, osteoporosis, macular degeneration, chronic obstructive pulmonary disease (COPD), glaucoma, obesity, fibrosis, cirrhosis, hepatic steatosis, and diabetes.

7. The method of claim 6, wherein the cancer is one or more of osteosarcoma, breast cancer, prostate cancer, colorectal cancer, lung cancer, melanoma, kidney cancer, lymphoma, uterine cancer, pancreatic cancer, non-melanoma skin cancer, and bladder cancer.

8. The method of claim 6, wherein the neurodegenerative disease is one or more of dementia, ataxia, Huntington's disease, a motor neuron disease, or a tau-mediated neurodegenerative disease, optionally Alzheimer's disease, Parkinson's disease, or progressive supranuclear palsy.

9. The method of claim 6, wherein the cardiovascular disease is one or more of atherosclerosis, idiopathic pulmonary fibrosis, coronary heart disease, congestive heart failure, coronary artery disease, peripheral arterial disease, valvular heart disease, arrhythmia, ischemic cardiomyopathy, hypertension, and stroke.

10. The method of claim 6, wherein the autoimmune disease is one or more of multiple sclerosis, Crohn's disease, rheumatoid arthritis, an antineutrophilic cytoplasmic antibody (ANCA) associated vasculitide, and systemic lupus erythematosus.

11. The method of claim 6, wherein the fibrosis is one or more of systemic sclerosis, scleroderma, idiopathic pulmonary fibrosis, and interstitial lung disease.

12. The method of any one of claims 1-11, wherein at least one of the antigenic proteins, if present, is a HCMV glycoprotein B.

13. The method of any one of claims 1-11, wherein at least one of the antigenic proteins, if present, comprises a sequence from HCMV glycoprotein B.

14. The method of claim 13, wherein the sequence from HCMV glycoprotein B is or comprises a truncated sequence of HCMV glycoprotein B.

15. The method of any one of claims 1-11, wherein each antigenic peptide, if present, comprises a sequence of 9-30 amino acids derived from a protein from a human 70 cytomegalovirus.

16. The method of claim 15, wherein each antigenic peptide, if present, comprises a sequence of 9-30 amino acids derived from HCMV glycoprotein B.

17. The method of any one of claims 1-16, wherein the commensal human cytomegaloviruses, if present, comprise one or more of the AD169, Towne, Toledo, PH, TR, FIX, VR1814, Merlin, and TB40/E HCMV strains.

18. The method of any one of claims 1-17, wherein the T cell adjuvant comprises a short chain fatty acid.

19. The method of claim 18, wherein the short chain fatty acid comprises butyrate, pentanoate, or a combination thereof.

20. The method of any one of claims 1-19, wherein the T cell adjuvant comprises one or more of nanoparticles that enhance T cell response, poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminum salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).

21. A composition comprising:
90 a plurality of (i) antigenic proteins from commensal human cytomegaloviruses (HCMV), (ii) antigenic peptides derived from proteins from commensal human cytomegaloviruses, or (iii) live or live-attenuated commensal human cytomegaloviruses; and
a T cell adjuvant that increases T cell response to the plurality of the antigenic 95 proteins, the antigenic peptides, or the live or live-attenuated commensal human cytomegaloviruses, for use in a method of treating, or reducing the risk of developing, an aging-associated disease or condition in a subject.

22. The composition of claim 1, wherein the aging-associated condition is one or more of graying hair, hearing loss, a cataract, frailty, and sarcopenia.

23. The composition of claim 1, wherein the aging-associated disease is one or more of a cancer, a cardiovascular disease, a neurodegenerative disease, a renal disease, an autoimmune disease, arthritis, osteoporosis, macular degeneration, chronic obstructive pulmonary disease (COPD), glaucoma, obesity, fibrosis, cirrhosis, hepatic steatosis, and diabetes.

24. The composition of any one of claims 21-23, wherein each antigenic protein, if present, is a HCMV glycoprotein B.

25. The composition of any one of claims 21-23, wherein each antigenic protein, if present, comprises a sequence from HCMV glycoprotein B.

26. The composition of claim 25, wherein the sequence from HCMV glycoprotein B is a truncated sequence of HCMV glycoprotein B.

27. The composition of any one of claims 21-23, wherein each antigenic peptide, if present, comprises a sequence of 9-30 amino acids derived from a protein from a human cytomegalovirus.

28. The composition of claim 27, wherein each antigenic peptide, if present, comprises a sequence of 9-30 amino acids derived from HCMV glycoprotein B.

29. The composition of any one of claims 21-28, wherein the commensal human cytomegaloviruses, if present, comprise one or more of the AD169, Towne, Toledo, PH, TR, FIX, VR1814, Merlin, and TB40/E HCMV strains.

30. The composition of any one of claims 21-29, wherein the T cell adjuvant comprises a short chain fatty acid.

31. The composition of claim 30, wherein the short chain fatty acid comprises butyrate, pentanoate, or a combination thereof.

32. The composition of any one of claims 21-29, wherein the T cell adjuvant comprises one or more of nanoparticles that enhance T cell response, poly-ICLC (carboxymethylcellulose, polyinosinic-polycytidylic acid, and poly-L-lysine double-stranded RNA), Imiquimods, CpG oligodeoxynuceotides and formulations (IC31, QB10), AS04 (aluminum salt formulated with 3-O-desacyl-4′-monophosphoryl lipid A (MPL)), AS01 (MPL and the saponin QS-21), MPLA, STING agonists, other TLR agonists, Candida albicans Skin Test Antigen (Candin), GM-CSF, Fms-like tyrosine kinase-3 ligand (Flt3L), and/or IFA (Incomplete Freund's adjuvant).
